(19) United States
(12) Patent Application Publication
(10) Pub. No.: US 2026/0258380 A1
(43) Pub. Date: Sep. 3, 2026

(54) Plastic-Degrading Enzymes and Methods of Identifying Same

(71) Applicant: The Board of Trustees of the Leland Stanford Junior University, Stanford, CA (US)

(72) Inventors: Nikita Khlystov, Stanford, CA (US); Jennifer R. Cochran, Stanford, CA (US)

(21) Appl. No.: 19/107,997

(22) PCT Filed: Sep. 1, 2023
(86) PCT No.: PCT/US2023/073353

§ 371 Date: Feb. 28, 2025

Related U.S. Application Data
(60) Provisional application No. 63/403,616, filed on Sep. 2, 2022.

(51) Int. Cl. C12N 9/18; C08J 11/10

(52) U.S. Cl. CPC C12N 9/18; C08J 11/105; C12Y 301/01074; C08J 2367/04

(57) ABSTRACT

Provided are methods of assessing an enzyme for plastic-degrading activity. In certain embodiments, the methods comprise contacting a plastic with the enzyme under conditions suitable for plastic-degrading enzyme activity, and assessing for degradation of the plastic by the enzyme. Also provided are plastic-degrading enzymes and methods of using the same. As a non-limiting example, the plastic-degrading enzymes are polyester-degrading enzymes. As an example, polylactic acid (PLA)-degrading enzymes and methods of using the same. As another example, polyethylene terephthalate (PET)-degrading enzymes and methods of using the same.

### CROSS-REFERENCE TO RELATED APPLICATIONS

This application claims the benefit of U.S. Provisional Patent Application No. 63/403,616, filed Sep. 2, 2022, which application is incorporated herein by reference in its entirety.

### INCORPORATION BY REFERENCE OF SEQUENCE LISTING PROVIDED AS A SEQUENCE LISTING XML FILE

A Sequence Listing is provided herewith as a Sequence Listing XML, “STAN-2021WO_SEQ_LIST”, created on Aug. 31, 2023, and having a size of 39,192 bytes. The contents of the Sequence Listing XML are incorporated herein by reference in their entirety.

### INTRODUCTION

Over 360 million tons of plastic waste is generated around the world each year. Over 90% of plastic waste cannot be recycled, resulting in increasing ecological damage as well as human health hazards globally. Raw resources and economic value are being sequestered at unsustainable rates in unrecyclable plastic-containing waste streams. Present-day plastic recycling methods are primarily limited by an inability to handle multi-material and/or physically complex plastic-containing waste streams. Moreover, plastics recycling facilities are currently centralized due to the low-margin, high-volume nature of this industry; this requires long-distance transportation of waste, constraining plastics recycling to only certain materials and geographical areas to maintain economical operation.

### SUMMARY

Provided are methods of assessing an enzyme for plastic-degrading activity. In certain embodiments, the methods comprise contacting a plastic with the enzyme under conditions suitable for plastic-degrading enzyme activity, and assessing for degradation of the plastic by the enzyme. Also provided are plastic-degrading enzymes and methods of using the same. As a non-limiting example, the plastic-degrading enzymes are polyester-degrading enzymes. As an example, polylactic acid (PLA)-degrading enzymes and methods of using the same. As another example, polyethylene terephthalate (PET)-degrading enzymes and methods of using the same.

### BRIEF DESCRIPTION OF THE DRAWINGS

FIG. 1. Characterization of PLA-containing nonwoven fabric treated with CLE1 (SEQ ID NO:1) by FTIR relative to untreated fabric and a polypropylene control.

FIG. 2. Characterization of residual PLA content of PLA-containing nonwoven fabric treated with CLE1 (SEQ ID NO:1) by FTIR relative to untreated fabric and a polypropylene control as calculated by the reduction of the absorbance peak at 1759 cm⁻¹ corresponding to C═O stretching relative to the control samples.

FIG. 3. Gravimetric analysis of PLA-containing nonwoven fabric treated with CLE1 (SEQ ID NO:1) relative to untreated fabric following solvent extraction of residual PLA.

### DETAILED DESCRIPTION

Before the methods and plastic-degrading enzymes of the present disclosure are described in greater detail, it is to be understood that the methods and plastic-degrading enzymes are not limited to particular embodiments described, as such may, of course, vary. It is also to be understood that the terminology used herein is for the purpose of describing particular embodiments only, and is not intended to be limiting, since the scope of the methods and plastic-degrading enzymes will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods and plastic-degrading enzymes. The upper and lower limits of these smaller ranges may independently be included in the smaller ranges and are also encompassed within the methods and plastic-degrading enzymes, subject to any specifically excluded limit in the stated range. Where the stated range includes one or both of the limits, ranges excluding either or both of those included limits are also included in the methods and plastic-degrading enzymes.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods and plastic-degrading enzymes belong. Although any methods and plastic-degrading enzymes similar or equivalent to those described herein can also be used in the practice or testing of the methods and plastic-degrading enzymes, representative illustrative methods and plastic-degrading enzymes are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods and plastic-degrading enzymes are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods and plastic-degrading enzymes, which are, for clarity, described in the context of separate embodiments, may also be provided in combination in a single embodiment. Conversely, various features of the methods and plastic-degrading enzymes, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and plastic-degrading enzymes and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

Herein, the terms “peptide”, “polypeptide”, “protein”, “enzyme” refer to a chain of amino acids linked by peptide bonds, regardless of the number of amino acids forming said chain. The amino acids are herein represented by their one-letter or three-letters code according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (He); K: lysine (Lys); L: leucine (Leu); M: methionine (Met); N: asparagine (Asn); P: proline (Pro); Q: glutamine (Gin); R: arginine (Arg); S: serine (Ser); T: threonine (Thr); V: valine (Val); W: tryptophan (Trp) and Y: tyrosine (Tyr).

The term “esterase” refers to an enzyme which belongs to a class of hydrolases classified as EC 3.1.1 according to Enzyme Nomenclature that catalyzes the hydrolysis of esters into an acid and an alcohol. The term “cutinase” refers to the esterases classified as EC 3.1.1.74 according to Enzyme Nomenclature that are able to catalyse the chemical reaction of production of cutin monomers from cutin and water.

The terms “wild-type” or “parent” refer to the non-mutated version of a polypeptide as it appears naturally. In the present case, the parent cutinase refers to the cutinase having the amino acid sequence as set forth in SEQ ID NO:3, SEQ ID NO:4, or SEQ ID NO:5.

The terms “mutant” and “variant” refer to polypeptides derived from SEQ ID NO:3, SEQ ID NO:4, or SEQ ID NO:5 and comprising at least one modification or alteration, i.e., a substitution, insertion, and/or deletion, at one or more (e.g., several) positions and having a polyester degrading activity. The variants may be obtained by various techniques. In particular, examples of techniques for altering the DNA sequence encoding the wild-type protein, include, but are not limited to, site-directed mutagenesis, random mutagenesis and synthetic oligonucleotide construction. Thus, the terms “modification” and “alteration” as used herein in relation to a particular position means that the amino acid in this particular position has been modified compared to the amino acid in this particular position in the wild-type protein.

A “substitution” means that an amino acid residue is replaced by another amino acid residue. In some embodiments, the term “substitution” refers to the replacement of an amino acid residue by another selected from the naturally-occurring standard 20 amino acid residues (G, P, A, V, L, I, M, C, F, Y, W, H, K, R, Q, N, E, D, S and T). The sign “+” indicates a combination of substitutions. In the present document, the following terminology is used to designate a substitution: Q1R denotes that amino acid residue (Glutamine, Q) at position 1 of the parent sequence is substituted by an Arginine (R). Q1P/R/S denotes that amino acid residue (Glutamine, Q) at position 1 of the parent sequence is substituted by one of the following amino acids: Proline (P), Arginine (R), or Serine (S).

Unless otherwise specified, the positions disclosed in the present application are numbered by reference to the amino acid sequence set forth in one of SEQ ID NOS. 3-35, as applicable.

As used herein, the term “sequence identity” or “identity” refers to the number (or fraction expressed as a percentage %) of matches (identical amino acid residues) between two polypeptide sequences. The sequence identity is determined by comparing the sequences when aligned so as to maximize overlap and identity while minimizing sequence gaps. In particular, sequence identity may be determined using any of a number of mathematical global or local alignment algorithms, depending on the length of the two sequences. Sequences of similar lengths are preferably aligned using a global alignment algorithm (e.g., Needleman and Wunsch algorithm; Needleman and Wunsch, 1970) which aligns the sequences optimally over the entire length, while sequences of substantially different lengths are preferably aligned using a local alignment algorithm (e.g. Smith and Waterman algorithm (Smith and Waterman, 1981) or Altschul algorithm (Altschul et al., 1997; Altschul et al., 2005)). Alignment for purposes of determining percent amino acid sequence identity can be achieved in various ways that are within the skill in the art, for instance, using publicly available computer software available on internet web sites such as http://blast.ncbi.nlm.nih.gov/or http://www.ebi.ac.uk/Tools/emboss/). Those skilled in the art can determine appropriate parameters for measuring alignment, including any algorithms needed to achieve maximal alignment over the full length of the sequences being compared. For purposes herein, % amino acid sequence identity values refers to values generated using the pair wise sequence alignment program EMBOSS Needle that creates an optimal global alignment of two sequences using the Needleman-Wunsch algorithm, wherein all search parameters are set to default values, i.e., Scoring matrix=BLOSUM62, Gap open=10, Gap extend=0.5, End gap penalty=false, End gap open=10 and End gap extend=0.5.

A “plastic” or “plastic-containing” object comprises a material, object, or compound comprising of one or more polymers. A “polymer” refers to a chemical compound or mixture of compounds whose structure is constituted of multiple monomers (repeat units) linked by covalent chemical bonds. Within the context of the invention, the term polymer includes natural or synthetic polymers, constituted of a single type of repeat unit (i.e., homopolymers) or of a mixture of different repeat units (i.e., copolymers or heteropolymers). “Oligomers” refer to molecules containing from 2 to about 20 monomers. In certain embodiments, “plastic” or “plastic-containing” object comprises a material, object, or compound comprising one or more types of polyester(s). The term “polyester-containing” refers to a product, such as plastic product, comprising at least one polyester in crystalline, semi-crystalline or totally amorphous forms. In a particular embodiment, the polyester containing material refers to any item made from at least one plastic material, such as plastic sheet, tube, rod, profile, shape, film, massive block, etc., which contains at least one polyester, and possibly other substances or additives, such as plasticizers, mineral or organic fillers. In another particular embodiment, the polyester containing material refers to a plastic compound, or plastic formulation, in a molten or solid state, suitable for making a plastic product. In another particular embodiment, the polyester containing material refers to textile, fabrics or fibers comprising at least one polyester. In another particular embodiment, the polyester containing material refers to plastic waste or fiber waste comprising at least one polyester.

The term “polyester(s)” encompasses but is not limited to polyethylene terephthalate (PET), polylactic acid (PLA), polytrimethylene terephthalate (PTT), polybutylene terephthalate (PBT), polyethylene isosorbide terephthalate (PEIT), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), poly(ethylene adipate) (PEA), polyethylene naphthalate (PEN) and combinations of any of these polymers.

A plastic substrate may comprise a material or chemical compound representative of a particular type of plastic. The plastic substrate may be in a form amenable to dispersion, suspension, and/or dissolution in solid growth media, for example as a solution, emulsion, solids, film, fibers, micronized powder, nanoparticles, or any combination of the preceding. The plastic substrate may contain one or more plastic types. The plastic substrate may contain one or more other non-plastic materials. In a non-limiting example, the plastic substrate may comprise one or more polyesters. The polyester may refer to any of the polyesters described above. In one embodiment described herein, the polyester is polylactic acid (PLA). In another embodiment described herein, the polyester is polyethylene terephthalate (PET). In a non-limiting example, the plastic substrate may be comprised of polyester and a non-polyester material such as cotton. The polyester-containing plastic substrate may include but is not limited to a consumer-grade Ingeo NatureWorks PLA plastic cup; PLA-based cutlery; a consumer PET water bottle; commercially available PLA or PET polymer of defined polydispersity and molecular weight; PLA or PET micronized powder; polyester-based spun fiber; polyester-containing textile material; oligomeric polyester compounds; bis(2-hydroxyethyl)terephthalate; terephthalic acid bis(2-hydroxyethyl)ester dibenzoate; terephthalic acid bis(2-hydroxyethyl)ester ditoluate; or terephthalic acid bis(2-hydroxyethyl)ester dianisate. The plastic substrate may also be comprised of polyamides such as nylon 6 and nylon 6,6, polystyrene, polyurethane, polyimides, polyethers, polycarbonates, polythenes such as polyethylene and polypropylene, other types of plastics, or any combination thereof that are amenable to degradation by enzymatic activity.

### Plastic-Degrading Enzymes

In one aspect, provided herein are enzymes that degrade a plastic, wherein the enzymes comprise one or more substitutions as compared to a wild-type enzyme. Non-limiting wild-type enzymes include those having SEQ ID NOS: 3, 4, and 5. Plastics include polyesters, such as PLA and PET. In example embodiments, enzymes having SEQ ID NOS: 3 or 5, or one or more substitutions thereof, degrade PLA. In example embodiments, enzymes having SEQ ID NO:4, or one or more substitutions thereof, degrade PET.

Non-limiting example enzymes include any one of SEQ ID NOS: 6-23, and enzymes comprising a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 6-23. The enzyme may degrade plastic. For example, the enzyme may degrade a polyester. The enzyme may degrade PET.

In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:4, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:6, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:7, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:8, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:9, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:10, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:11, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:12, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:13, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:14, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:15, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:16, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:17, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:18, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:19, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:20, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:21, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:22, wherein the enzyme comprises arginine at position 1, serine at position 3, lysine at position 29, and glutamine at position 47. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:23, wherein the enzyme comprises arginine at position 1, serine at position 3, lysine at position 29, and glutamine at position 47. The enzymes may degrade plastic. For example, the enzyme may degrade a polyester. The enzyme may degrade PET. A non-limiting example enzyme comprises SEQ ID NO:4, or a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:4. The enzyme may degrade plastic. For example, the enzyme may degrade a polyester. The enzyme may degrade PET. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:4, wherein the enzyme comprises a substitution at position 1, according to SEQ ID NO:4. The amino acid substitution at position 1 may be R, A, C, D, E, F, G, H, K, L, M, N, P, S, T, V, or Y. In a non-limiting example embodiment, the amino acid substitution at position 1 is R. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:4, wherein the enzyme comprises a substitution at position 3, according to SEQ ID NO:4. The amino acid substitution at position 3 may be S, A, C, D, E, F, H, I, L, N, Q, R, T, V, or Y. In a non-limiting example embodiment, the amino acid substitution at position 3 is S. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:4, wherein the enzyme comprises a substitution at position 29, according to SEQ ID NO:4. The amino acid substitution at position 29 may be K. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:4, wherein the enzyme comprises a substitution at position 47, according to SEQ ID NO:4. The amino acid substitution at position 47 may be Q, A, C, F, G, H, I, L, M, N, R, S, T, V, or Y. In a non-limiting example embodiment, the amino acid substitution at position 47 is Q. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:4, wherein the enzyme comprises a substitution at position 4, according to SEQ ID NO:4. The amino acid substitution at position 4 may be V. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:4, wherein the enzyme comprises a substitution at position 11, according to SEQ ID NO:4. The amino acid substitution at position 11 may be N. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:4, wherein the enzyme comprises a substitution at position 15, according to SEQ ID NO:4. The amino acid substitution at position 15 may be D. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:4, wherein the enzyme comprises a substitution at position 37, according to SEQ ID NO:4. The amino acid substitution at position 37 may be A, I, L, M, N, P, S, or V. In a non-limiting example embodiment, the amino acid substitution at position 37 is L. In a non-limiting example embodiment, the amino acid substitution at position 37 is I. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:4, wherein the enzyme comprises a substitution at position 164, according to SEQ ID NO:4. The amino acid substitution at position 164 may be S. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:4, wherein the enzyme comprises a substitution at position 166, according to SEQ ID NO:4. The amino acid substitution at position 166 may be A. A non-limiting example combination of substitutions includes substitutions at positions Q1, G3, T29, and E47. The amino acid substitution at position 1 may be R, A, C, D, E, F, G, H, K, L, M, N, P, S, T, V, or Y. In a non-limiting example embodiment, the amino acid substitution at position is R. The amino acid substitution at position 3 may be S, A, C, D, E, F, H, I, L, N, Q, R, T, V, or Y. In a non-limiting example embodiment, the amino acid substitution at position is S. The amino acid substitution at position 29 may be K. The amino acid substitution at position 47 may be Q, A, C, F, G, H, I, L, M, N, R, S, T, V, or Y. In a non-limiting example embodiment, the amino acid substitution at position 47 is Q.

A non-limiting example enzyme comprises any one of SEQ ID NOS: 6-23, or a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 6-23. The enzyme may degrade plastic. For example, the enzyme may degrade a polyester. The enzyme may degrade PET. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 6-23, wherein the enzyme comprises a substitution at position 1, according to any one of SEQ ID NOS: 6-23. The amino acid substitution at position 1 may be R, A, C, D, E, F, G, H, K, L, M, N, P, S, T, V, or Y. In a non-limiting example embodiment, the amino acid substitution at position is R. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 6-23, wherein the enzyme comprises a substitution at position 3, according to any one of SEQ ID NOS: 6-23. The amino acid substitution at position 3 may be S, A, C, D, E, F, H, I, L, N, Q, R, T, V, or Y. In a non-limiting example embodiment, the amino acid substitution at position is S. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 6-23, wherein the enzyme comprises a substitution at position 29, according to any one of SEQ ID NOS: 6-23. The amino acid substitution at position 29 may be K. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 6-23, wherein the enzyme comprises a substitution at position 47, according to any one of SEQ ID NOS: 6-23. The amino acid substitution at position 47 may be Q, A, C, F, G, H, I, L, M, N, R, S, T, V, or Y. In a non-limiting example embodiment, the amino acid substitution at position 47 is Q. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 6-23, wherein the enzyme comprises a substitution at position 4, according to any one of SEQ ID NOS: 6-23. The amino acid substitution at position 4 may be V. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 6-23, wherein the enzyme comprises a substitution at position 11, according to any one of SEQ ID NOS: 6-23. The amino acid substitution at position 11 may be N. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 6-23, wherein the enzyme comprises a substitution at position 15, according to any one of SEQ ID NOS: 6-23. The amino acid substitution at position 15 may be D. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 6-23, wherein the enzyme comprises a substitution at position 37, according to any one of SEQ ID NOS: 6-23. The amino acid substitution at position 37 may be A, I, L, M, N, P, S, or V. In a non-limiting example embodiment, the amino acid substitution at position 37 is L. In a non-limiting example embodiment, the amino acid substitution at position 37 is I. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 6-23, wherein the enzyme comprises a substitution at position 164, according to any one of SEQ ID NOS: 6-23. The amino acid substitution at position 164 may be S. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 6-23, wherein the enzyme comprises a substitution at position 166, according to any one of SEQ ID NOS: 6-23. The amino acid substitution at position 166 may be A. A non-limiting example combination of substitutions includes substitutions at positions Q1, G3, T29, and E47. The amino acid substitution at position 1 may be R, A, C, D, E, F, G, H, K, L, M, N, P, S, T, V, or Y. In a non-limiting example embodiment, the amino acid substitution at position is R. The amino acid substitution at position 3 may be S, A, C, D, E, F, H, I, L, N, Q, R, T, V, or Y. In a non-limiting example embodiment, the amino acid substitution at position is S. The amino acid substitution at position 29 may be K. The amino acid substitution at position 47 may be Q, A, C, F, G, H, I, L, M, N, R, S, T, V, or Y. In a non-limiting example embodiment, the amino acid substitution at position 47 is Q.

Additional non-limiting example enzymes provided herein include any one of SEQ ID NOS: 24-35, and enzymes comprising a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 24-35. The enzyme may degrade plastic. For example, the enzyme may degrade a polyester. The enzyme may degrade PLA.

In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:3, wherein the enzyme comprises valine at position 40, lysine at position 112, and arginine at position 191. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:5, wherein the enzyme comprises valine at position 40, lysine at position 112, and arginine at position 191. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:24, wherein the enzyme comprises valine at position 40, lysine at position 112, and arginine at position 191. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:25, wherein the enzyme comprises valine at position 40, lysine at position 112, and arginine at position 191. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:26, wherein the enzyme comprises valine at position 40, lysine at position 112, and arginine at position 191. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:27, wherein the enzyme comprises valine at position 40, lysine at position 112, and arginine at position 191. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:28, wherein the enzyme comprises valine at position 40, lysine at position 112, and arginine at position 191. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:29, wherein the enzyme comprises valine at position 40, lysine at position 112, and arginine at position 191. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:30, wherein the enzyme comprises valine at position 40, lysine at position 112, and arginine at position 191. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:31, wherein the enzyme comprises valine at position 40, lysine at position 112, and arginine at position 191. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:32, wherein the enzyme comprises valine at position 40, lysine at position 112, and arginine at position 191. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:33, wherein the enzyme comprises valine at position 40, lysine at position 112, and arginine at position 191. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:34, wherein the enzyme comprises valine at position 40, lysine at position 112, and arginine at position 191. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:35, wherein the enzyme comprises valine at position 40, lysine at position 112, and arginine at position 191.

A non-limiting example enzyme comprises SEQ ID NO:3, or a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:3. The enzyme may degrade plastic. For example, the enzyme may degrade a polyester. The enzyme may degrade PLA. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:3, wherein the enzyme comprises a substitution at position 40, according to SEQ ID NO:3. The amino acid substitution at position 40 may be V. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:3, wherein the enzyme comprises a substitution at position 112, according to SEQ ID NO:3. The amino acid substitution at position 112 may be R. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:3, wherein the enzyme comprises a substitution at position 191, according to SEQ ID NO:3. The amino acid substitution at position 191 may be V. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:3, wherein the enzyme comprises a substitution at position 94, according to SEQ ID NO:3. The amino acid substitution at position 94 may be F. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:3, wherein the enzyme comprises a substitution at position 111, according to SEQ ID NO:3. The amino acid substitution at position 111 may be K. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:3, wherein the enzyme comprises a substitution at position 118, according to SEQ ID NO:3. The amino acid substitution at position 118 may be P. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:3, wherein the enzyme comprises a substitution at position 148, according to SEQ ID NO:3. The amino acid substitution at position 148 may be I. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:3, wherein the enzyme comprises a substitution at position 220, according to SEQ ID NO:3. The amino acid substitution at position 220 may be E. A non-limiting example combination of substitutions includes substitutions at positions 40, 112, and 191. The amino acid substitution at position 40 may be V. The amino acid substitution at position 112 may be R. The amino acid substitution at position 191 may be V.

A non-limiting example enzyme comprises SEQ ID NO:5, or a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:5. The enzyme may degrade plastic. For example, the enzyme may degrade a polyester. The enzyme may degrade PLA. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:5, wherein the enzyme comprises a substitution at position 40, according to SEQ ID NO:5. The amino acid substitution at position 40 may be V. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:5, wherein the enzyme comprises a substitution at position 112, according to SEQ ID NO:5. The amino acid substitution at position 112 may be R. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:5, wherein the enzyme comprises a substitution at position 191, according to SEQ ID NO:5. The amino acid substitution at position 191 may be V. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:5, wherein the enzyme comprises a substitution at position 94, according to SEQ ID NO:5. The amino acid substitution at position 94 may be F. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:5, wherein the enzyme comprises a substitution at position 111, according to SEQ ID NO:5. The amino acid substitution at position 111 may be K. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:5, wherein the enzyme comprises a substitution at position 118, according to SEQ ID NO:5. The amino acid substitution at position 118 may be P. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:5, wherein the enzyme comprises a substitution at position 148, according to SEQ ID NO:5. The amino acid substitution at position 148 may be I. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to SEQ ID NO:5, wherein the enzyme comprises a substitution at position 220, according to SEQ ID NO:5. The amino acid substitution at position 220 may be E. A non-limiting example combination of substitutions includes substitutions at positions 40, 112, and 191. The amino acid substitution at position 40 may be V. The amino acid substitution at position 112 may be R. The amino acid substitution at position 191 may be V.

A non-limiting example enzyme comprises any one of SEQ ID NOS: 24-35, or a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 24-35. The enzyme may degrade plastic. For example, the enzyme may degrade a polyester. The enzyme may degrade PLA. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 24-35, wherein the enzyme comprises a substitution at position 40, according to any one of SEQ ID NOS: 24-35. The amino acid substitution at position 40 may be V. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 24-35, wherein the enzyme comprises a substitution at position 112, according to any one of SEQ ID NOS: 24-35. The amino acid substitution at position 112 may be R. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 24-35, wherein the enzyme comprises a substitution at position 191, according to any one of SEQ ID NOS: 24-35. The amino acid substitution at position 191 may be V. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 24-35, wherein the enzyme comprises a substitution at position 94, according to any one of SEQ ID NOS: 24-35. The amino acid substitution at position 94 may be F. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 24-35, wherein the enzyme comprises a substitution at position 111, according to any one of SEQ ID NOS: 24-35. The amino acid substitution at position 111 may be K. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 24-35, wherein the enzyme comprises a substitution at position 118, according to any one of SEQ ID NOS: 24-35. The amino acid substitution at position 118 may be P. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 24-35, wherein the enzyme comprises a substitution at position 148, according to any one of SEQ ID NOS: 24-35. The amino acid substitution at position 148 may be I. In some embodiments, the enzyme comprises a sequence at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identical to any one of SEQ ID NOS: 24-35, wherein the enzyme comprises a substitution at position 220, according to any one of SEQ ID NOS: 24-35. The amino acid substitution at position 220 may be E. A non-limiting example combination of substitutions includes substitutions at positions 40, 112, and 191. The amino acid substitution at position 40 may be V. The amino acid substitution at position 112 may be R. The amino acid substitution at position 191 may be V.

### Nucleic Acids, Expression Constructs, Cells and Compositions

Aspects of the present disclosure further include nucleic acids and expression constructs. For example, provided are nucleic acids encoding any of the enzymes of the present disclosure.

The nucleotide sequences of the nucleic acids of the present may be codon-optimized. “Codon-optimized” refers to changes in the codons of the polynucleotide encoding a polypeptide to those preferentially used in a particular organism such that the encoded protein is efficiently expressed in the organism of interest. Although the genetic code is degenerate in that most amino acids are represented by several codons, called “synonyms” or “synonymous” codons, codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, a nucleic acid of the present disclosure encoding a polypeptide may be codon-optimized for optimal production from the host organism selected for expression, e.g., bacterial cells, such as E. coli cells or yeast cells, such as, Saccharomyces cerevisiae.

Also provided are expression constructs comprising any of the nucleic acids of the present disclosure. As used herein, an “expression construct” is a circular or linear polynucleotide (a polymer composed of naturally occurring and/or non-naturally occurring nucleotides) comprising a region that encodes a polypeptide of the present disclosure, operably linked to a suitable promoter, e.g., a constitutive or inducible promoter. In some embodiments, expression of the polypeptide is under the control of one or more heterologous regulatory elements, e.g., promoter, enhancer, etc., present in the expression construct. In some embodiments, expression of the polypeptide may be controlled by one or more endogenous regulatory elements, e.g., promoter, enhancer, etc., at or near a genomic locus into which the expression construct is inserted.

The expression constructs (e.g., vectors) can be suitable for replication and integration in prokaryotes, eukaryotes, or both. The expression constructs may contain functionally appropriately oriented transcription and translation terminators, initiation sequences, and promoters useful for regulation of the expression of the nucleic acid encoding the enzymes. The expression constructs optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid one may construct expression constructs which typically contain a strong promoter to direct transcription, a ribosome binding site for translational initiation, and a transcription/translation terminator, each in functional orientation to each other and to the protein-encoding sequence. Examples of regulatory regions suitable for this purpose in E. coli are the promoter and operator region of the E. coli tryptophan biosynthetic pathway, the leftward promoter of phage lambda (P_{L}), and the L-arabinose (araBAD) operon. The inclusion of selection markers in DNA vectors transformed in E. coli is also useful. Examples of such markers include genes specifying resistance to ampicillin, tetracycline, or chloramphenicol. Expression systems for expressing the enzyme are available for use in, for example, E. coli, Bacillus sp. and Salmonella. E. coli systems may also be used. Transducing cells with nucleic acids (e.g., expression constructs) can involve, for example, incubating lipidic microparticles containing nucleic acids with cells or incubating viral vectors containing nucleic acids with cells.

In certain embodiments, upon delivery of an expression construct to cells, one or more of the expression constructs are episomal (e.g., extra-chromosomal), where by “episome” or “episomal” is meant a polynucleotide that replicates independently of the cell's chromosomal DNA. A non-limiting example of an episome that may be employed is a plasmid.

Aspects of the present disclosure further include cells comprising a nucleic acid of the present disclosure, as well as cells comprising an expression construct of the present disclosure. In certain embodiments, the cells are prokaryotic cells (e.g., bacteria, E. coli), yeast cells (e.g., Saccharomyces species, e.g., Saccharomyces cerevisiae), insect (e.g., drosophila) cells, amphibian (e.g., frog, e.g., Xenopus) cells, plant cells, etc. According to some embodiments, the cells are mammalian cells. Mammalian cells of interest include human cells, rodent cells, and the like and include cell lines, such as, CHO cells, HEK293 cells, etc. In a particular embodiment, the cells of interest are Saccharomyces cerevisiae. In another particular embodiment, the cells of interest are Escherichia coli.

Also provided by the present disclosure are compositions. According to some embodiments, provided are compositions comprising any of the cells, polypeptides, nucleic acids, and/or expression constructs of the present disclosure.

Such compositions may comprise the polypeptides, nucleic acids, expression constructs, and/or cells present in a liquid medium. The liquid medium may be an aqueous liquid medium, such as water, a buffered solution, or the like. In certain embodiments, the liquid medium is a cell culture medium. The cell culture medium may be bacterial or yeast cell culture mediums. Non-limiting examples of cell culture media include peptone, yeast extract, and dextrose or glucose; Minimal Essential Media, DMEM, a-MEM, RPMI Media, Clicks, F-12, X-Vivo 15, X-Vivo 20, Optimizer, and the like.

In certain embodiments, the compositions may be frozen or lyophilized. In certain embodiments, the cell(s), cell extract(s), polypeptide(s), and/or enzyme(s) of the present disclosure may be formulated into suspensions, sprayable solutions, hydrogels, or otherwise easily dispersible formulations.

In certain embodiments, the composition may be liquid or dry, for instance in the form of a powder. In certain embodiments, the composition is a lyophilizate. The composition may further comprise excipients and/or reagents. Appropriate excipients encompass buffers commonly used in biochemistry, and/or agents for adjusting pH. The composition may be obtained by mixing the cell(s), cell extract(s), polypeptide(s), and/or enzyme(s) of the present disclosure with one or several excipients.

In certain embodiments, the composition may further comprise additional cell(s), cell extract(s), enzyme(s), and/or polypeptide(s) exhibiting an enzymatic activity. In certain embodiments, the enzymatic activity is a plastic-degrading activity. The amounts of cell(s), cell extract(s), enzyme(s), and/or polypeptide(s) may be adapted depending e.g., on the nature of the plastic to degrade and/or the additional cell(s), cell extract(s), enzyme(s), and/or polypeptide(s) contained in the composition.

In certain embodiments, the composition is solubilized in an aqueous medium together with one or several excipients, especially excipients which are able to stabilize or protect the plastic-degrading cell(s), cell extract(s), enzyme(s), and/or polypeptide(s) from degradation. For instance, the cell(s), cell extract(s), enzyme(s), and/or polypeptide(s) of the present disclosure may be solubilized in water, eventually with additional components. The resulting mixture may then be dried so as to obtain a powder. Methods for drying such mixture are well known to the one skilled in the art and include, without limitation, lyophilization, freeze-drying, spray-drying, supercritical drying, down-draught evaporation, thin-layer evaporation, centrifugal evaporation, conveyer drying, fluidized bed drying, drum drying or any combination thereof.

### Production of Plastic-Degrading Enzymes

A method of producing the enzymes of the present disclosure is provided. The method may include, expressing a nucleic acid encoding the enzyme and optionally recovering the enzyme.

In an exemplary embodiment, an in vitro method of producing an enzyme of the present disclosure may include (a) contacting a nucleic acid, cassette or vector encoding the enzyme with an in vitro expression system; and (b) recovering the enzyme produced. In vitro expression systems are well-known by the person skilled in the art and are commercially available.

In another embodiment, the method of production comprises (a) culturing a cell that comprises a nucleic acid encoding an enzyme of the present disclosure under conditions suitable to express the nucleic acid; and optionally (b) recovering said enzyme from the cell culture.

Exemplary cells include recombinant Bacillus, recombinant E. coli, recombinant Aspergillus, recombinant Trichoderma, recombinant Streptomyces, recombinant Saccharomyces, recombinant Pichia, recombinant Vibrio or recombinant Yarrowia.

The cells are cultivated in a nutrient medium suitable for production of polypeptides. For example, the cell may be cultivated by shake flask cultivation, or small-scale or large-scale fermentation (including continuous, batch, fed-batch, or solid-state fermentations) in laboratory or industrial fermenters performed in a suitable medium and under conditions allowing the enzyme to be expressed and/or isolated. The cultivation can take place in a suitable nutrient medium, from commercial suppliers or prepared according to published compositions (e.g., in catalogs of the American Type Culture Collection).

In certain embodiments, the enzyme is excreted into the nutrient medium and is recovered directly from the culture supernatant. Alternatively, the enzyme can be recovered from cell lysates or after permeabilization. The enzyme may be recovered from the nutrient medium by procedures including, but not limited to, collection, centrifugation, filtration, extraction, spray-drying, evaporation, or precipitation. Optionally, the enzyme may be partially or totally purified by a variety of procedures including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction to obtain substantially pure polypeptides.

The plastic-degrading enzyme may be used as such, in purified form, either alone or in combinations with additional enzymes, to catalyze enzymatic reactions involved in the degradation and/or recycling of plastic(s) and/or plastic-containing material. In certain embodiments, the plastic to be degraded or recycled is a polyester or polyester-containing material or product. The plastic-degrading enzyme may be in soluble form, or on solid phase. In certain embodiments, it may be bound to cell membranes or lipid vesicles, or to synthetic supports such as glass, plastic, polymers, filter, membranes, for example in the form of beads, columns, and/or plates. In certain embodiments, the plastic-degrading enzyme may be in obtained as cell culture or cell extract derived from recombinant cells expressing, secreting, displaying, or exporting the plastic-degrading enzyme.

### Methods of Assessing Plastic-Degrading Activity

Aspects of the present disclosure include assessing the plastic-degrading activity of the polypeptides and cells of the present disclosure.

High-throughput screening of plastic-degrading activity may be performed using any device that involves an array having a plurality of physically separate wells or containers for liquid, for example microtiter spectroscopic plates or microcapillary arrays, where the wells or containers may have closed ends or open ends. Screening may also be performed using devices employing both liquids and solids, such as deposition of a single liquid mixture of plastic-degrading enzymes and/or a single liquid suspension of cells encoding, expressing, secreting, displaying, and/or exporting plastic-degrading enzymes on a homogeneous plastic substrate, and/or deposition of individual liquid volumes containing and/or encapsulating individual plastic-degrading enzymes or cells encoding, expressing, secreting, displaying, and/or exporting plastic-degrading enzymes on said homogeneous plastic substrate, where the plastic substrate may be comprised of a solid film of a specific type of plastic; a solid sheet of a specific type of plastic; and/or a microscopically heterogeneous but macroscopically homogeneous plastic-containing substrate such as fibers, fabrics, and/or textiles consisting of one or more material types (e.g., polyester, cotton, nylon, elastane). Embodiments of the screening methods described herein may be used in tandem and/or in combination with other embodiments of screening methods including those described herein to produce datasets enabling the identification and characterization of enzymes with improved plastic-degrading capabilities.

Libraries of plastic-degrading enzymes may be generated via random, site-directed, or site-saturation mutagenesis. For example, mutagenesis PCR may be performed using DNA base pair orthologs, using an error-prone DNA polymerase (e.g., GeneMorph II, Agilent Technologies), or using degenerate primers for site-saturation mutagenesis. The products of this PCR reaction may have homology of 10 to 50 base-pairs to a vector that allows for the expression, secretion, display, and/or export of the gene product out of the cell. Homologous recombination of the expression vector and the insert library may be achieved by transformation into yeast and subsequent selection for transformants via an appropriate selection marker. Variant libraries may be produced in other host organisms using appropriate transformation and assembly methods.

Halo-based screening may be used for identifying transformants encoding, secreting, or displaying enzymes with plastic-degrading capabilities. This may be achieved using plastic-containing agar growth plates that simultaneously enable organism growth, production of enzyme, and indication of plastic degradation. Recombinant organisms encoding, expressing, secreting, displaying, and/or exporting one or more plastic-degrading enzymes may be comprised of organisms of bacterial or fungal origin, for example Escherichia coli, Saccharomyces cerevisiae, Pichia pastoris, etc. Growth plates may be prepared by incorporating the plastic substrate with tryptone, peptone, yeast extract, salts, and other growth medium suitable for the culture of recombinant organisms capable of producing plastic-degrading enzymes. Plastic-degrading activity may be assayed by visual inspection, photography, image processing, or other means of identifying and characterizing halo production.

Screening for plastic-degrading activity may also be achieved using a suspension, mixture, emulsion, gel, sol, or any combination of the preceding of plastic-containing material in a liquid medium such as water or buffer without use of growth media components. Screening of plastic-degrading activity may be achieved in combination with halo-based activity screening. The plastic-containing suspension, mixture, emulsion, gel, sol, or any combination of the preceding may consist of a buffering agent with appropriate buffering capacity at the desired pH corresponding to optimal activity of the plastic-degrading enzyme, for example pH 7, 8, or 9 for polyester-degrading enzymes. Surfactants, dispersants, emulsifiers, and other compounds may be used to achieve homogeneous dispersion of the plastic-containing material. The plastic-containing material may be in the form of solids, film, fibers, micronized powder, nanoparticles, or any combination of the preceding. Recombinant organisms encoding plastic-degrading enzyme(s), extracts thereof containing plastic-degrading enzymes, or other samples containing plastic-degrading enzyme(s) may be prepared in high-throughput devices, for example microtiter plates, 96-deep-well plates, or microcapillary arrays, as to contain sufficient quantities of enzyme for activity detection. In one non-limiting set of examples, the cell culture, cell extract, cell lysate, or culture medium depleted of cells is brought in contact with an appropriate plastic-containing material in the context of high-throughput devices.

Enzymes with plastic-degrading activity may be identified by extracting the DNA vector containing the gene encoding the plastic-degrading enzyme and sequencing the relevant part or whole of the DNA vector by, for example, Sanger DNA sequencing or Illumina next-generation sequencing, to identify advantageous mutations. Plastic-degrading enzymes may be expressed in recombinant organisms to produce sufficient quantities of plastic-degrading enzyme for activity analysis. Plastic-degrading enzymes may be purified using methods including but not limited to affinity chromatography, size exclusion chromatography, and/or ion exchange chromatography, to enable and/or facilitate activity analysis.

In some embodiments, the purified enzyme variants are tested on plastic-containing materials and assessed by an appropriate method to calculate, quantify, or otherwise characterize plastic-degrading activity. Plastic-degrading activity may be assessed using a method such as fluorescence, gravimetry, liquid chromatography mass spectroscopy (LCMS), or Fourier transform infrared spectroscopy (FTIR). In some embodiments, the plastic is a polyester, such as polyester solid, film, fiber, post-consumer multi-material textile waste containing for example cotton and polyester. In some embodiments, the polyester is selected from PET or PLA or a combination thereof.

In certain embodiments, plastic-degrading activity is assessed using a spectroscopic method, for example optical density, colorimetry, optical absorbance, or fluorescence. In a particular embodiment, PET-degrading activity is assessed using fluorescence and calculated as the fluorescence increase before and after contact with a PET-degrading enzyme.

In certain embodiments, plastic-degrading activity is assessed using gravimetry and calculated as the mass change of a plastic-containing material before and after contact with a plastic-degrading enzyme. In certain embodiments, plastic-degrading activity is assessed using gravimetry and calculated as the mass change of a plastic-containing material after being contacted with a plastic-degrading enzyme relative to a plastic-containing material without being contacted with a plastic-degrading enzyme. In a particular embodiment, PLA-degrading activity is assessed using gravimetry and calculated as the mass change of a PLA-containing fabric before and after contact with a PLA-degrading enzyme and relative to a PLA-containing fabric without being contacted by PLA-degrading enzyme.

In certain embodiments, plastic-degrading activity is assessed using Fourier transform infrared spectroscopy (FTIR) and calculated as the change in the absorbance measurement at one or more wavelengths representative of the plastic-containing material to be degraded. In a particular embodiment, PLA-degrading activity is assessed using Fourier transform infrared spectroscopy (FTIR) and calculated as the change in the absorbance at 1759 cm⁻¹ representing the C═O bond in a PLA-containing material after contacting the PLA-containing material with a PLA-degrading enzyme.

In certain embodiments, plastic-degrading activity is assessed using liquid chromatography mass spectroscopy (LCMS) as the quantity of one or more products caused by plastic-degrading activity, such as a monomer, oligomer, or polymer, where the quantity may be the arithmetic sum of multiple products. In a particular embodiment, PET-degrading activity is assessed using LCMS as the quantity of ethylene glycol, terephthalate, mono-2-hydroxyethyl terephthalate (MHET), bis(2-hydroxyethyl) terephthalate (BHET), 1-(2-hydroxyethyl)-4-methyl terephthalate (HEMT), and/or dimethyl terephthalate (DMT), or any sum of monomers and oligomers, produced after contacting the PET-containing material with a PET-degrading enzyme.

### Methods of Use

Aspects of the present disclosure further include methods of using the polypeptides and cells of the present disclosure.

In certain embodiments, the polypeptides and cells of the present disclosure may be used in a method of degrading a plastic. The method may comprise contacting a plastic with a plastic-degrading enzyme as provided herein under conditions suitable for degradation of the plastic by the plastic-degrading enzyme. The contacting of the plastic with the plastic-degrading enzyme may include directly contacting the enzyme to the plastic, contacting the plastic with cells that express, secrete, display, or export the plastic-degrading enzyme, or containing the plastic with a cell extract thereof.

In certain embodiments, the polyester targeted by the plastic-degrading enzyme is selected from polyethylene terephthalate (PET), polytrimethylene terephthalate (PTT), polybutylene terephthalate (PBT), polyethylene isosorbide terephthalate (PEIT), polylactic acid (PLA), polyhydroxyalkanoate (PHA), polybutylene succinate (PBS), polybutylene succinate adipate (PBSA), polybutylene adipate terephthalate (PBAT), polyethylene furanoate (PEF), polycaprolactone (PCL), poly(ethylene adipate) (PEA), polyethylene naphthalate (PEN) and/or a combination of any of these polymers, preferably PET and/or PLA.

In a preferred embodiment, the polyester is PET, and at least monomers such as ethylene glycol or terephthalic acid, and/or oligomers such as mono-2-hydroxyethyl terephthalate (MHET), bis(2-hydroxyethyl) terephthalate (BHET), 1-(2-hydroxyethyl)-4-methyl terephthalate (HEMT), and/or dimethyl terephthalate (DMT) are recovered.

In another preferred embodiment, the polyester is PLA, and at least monomers such as lactic acid, and/or oligomers such as dilactate, trilactate, etc., are recovered.

The plastic-degrading enzyme may be purified, partially purified, or provided as cell culture supernatant, cell extract, and/or cell lysate. In certain embodiments, the method may include degrading plastic material under aerobic or anaerobic conditions. In certain embodiments, the plastic-containing material to be degraded contains a polyester. In certain embodiments, the polyester is PLA. The conditions suitable for degradation of the PLA can include a temperature between 0° C. and 90° C., preferably between 20° C. and 60° C., or more preferably between 25° C. and 40° C. In a particular embodiment, the degrading process is implemented at 37° C. In certain embodiments, the polyester is PET. The conditions suitable for degradation of the PET can include a temperature between 0° C. and 90° C., preferably between 37° C. and 75° C., or more preferably between 50° C. and 68° C. In a particular embodiment, the degrading process is implemented at 50° C. In another particular embodiment, the degrading process is implemented at 60° C. In another particular embodiment, the degrading process is implemented at 68° C.

More generally, the temperature is maintained below an inactivating temperature corresponding to the temperature at which the plastic-degrading enzyme is inactivated, for example at which the plastic-degrading enzyme has lost more than 80% of activity as compared to its activity at its optimum temperature and/or the recombinant organism is unable to express, produce, secrete, display, and/or export the plastic-degrading enzyme. Preferably, the temperature is maintained below the crystallization temperature (Tc) of the targeted plastic, more preferably below the onset of crystallization of the targeted plastic, and more preferably below the glass transition temperature (Tg) of the targeted plastic.

The time required for degrading a plastic-containing material may vary depending on the plastic-containing material itself, for example the nature and origin of the plastic-containing material, its composition, and shape; the type and amount of plastic-degrading enzyme used; and process parameters and conditions such as temperature, pH, applied forces, and additional agents. The process parameters may be adapted to the plastic-containing material and the envisioned degradation time.

In certain embodiments, the method is implemented in a continuous flow process, at a temperature at which the enzyme or cells producing the enzyme can be used several times and/or recycled. In certain embodiments, the enzyme is immobilized on or bound to synthetic supports, for example, glass, plastic, polymers, filter, membranes, in the form of, for example, substrates, surfaces, particles, beads, columns, and/or plates. In certain embodiments, enzyme immobilization is achieved by chemical modification such as chemical crosslinking, non-covalent bonding or interaction(s), and/or physical adhesion and/or other interaction(s).

In certain embodiments, the method is implemented at a pH comprised between 5 and 11, e.g., at a pH between 6 and 9, at a pH between 6.5 and 9, or at a pH between 6.5 and 8.

In certain embodiments, the plastic material, for example, a PLA-containing material and/or a PET-containing material, may be pretreated prior to contacting with the enzyme, in order to physically change its structure, so as to increase the surface of contact between the plastic and the enzyme or to increase the activity of the enzyme acting on the plastic.

In certain embodiments, monomers and/or oligomers are produced from a plastic-containing material, comprising of exposing a plastic-containing material to an enzyme, or corresponding cell or extract thereof, or composition, and optionally recovering monomers and/or oligomers. In certain embodiments, the plastic-containing material is a PET-containing material. In certain embodiments, the plastic-containing material is a PLA-containing material. Advantageously, the method can be used for plastic-containing materials comprising multiple types of materials. In certain embodiments, such a material is a polyester-containing blended textile. In certain embodiments, the polyester-containing blended textile also contains a cellulose-based material, for example cotton, viscose, and/or other manmade cellulosic fiber. In certain embodiments, the polyester-containing blended textile also contains an elastane-based material. In certain embodiments, the polyester-containing blended textile also contains a polyamide material, for example nylon 6 and/or nylon 6,6. Monomers and/or oligomers resulting from the depolymerization may be recovered, sequentially, or continuously. A single type of monomer and/or oligomer or several different types of monomers and/or oligomers may be recovered, depending on the starting plastic-containing material. The method is particularly useful for producing monomers selected from ethylene glycol and terephthalic acid, and/or oligomers selected from mono-2-hydroxyethyl terephthalate (MHET), bis(2-hydroxyethyl) terephthalate (BHET), 1-(2-hydroxyethyl)-4-methyl terephthalate (HEMT) and dimethyl terephthalate (DMT), from PET, and/or PET-containing plastic product. The method is particularly useful for producing monomers selected from lactic acid and lactate, and/or dilactate, from PLA, and/or PLA-containing plastic product.

The recovered monomers and/or oligomers may be further purified, using all suitable purifying methods and conditioned in a re-polymerizable form. Examples of purifying methods include stripping process, separation by aqueous solution, steam selective condensation, filtration and concentration of the medium after the bioprocess, separation, distillation, vacuum evaporation, extraction, electrodialysis, adsorption, ion exchange, precipitation, crystallization, concentration and acid addition dehydration and precipitation, nanofiltration, acid catalyst treatment, semi-continuous mode distillation or continuous-mode distillation, solvent extraction, evaporative concentration, evaporative crystallization, liquid/liquid extraction, hydrogenation, azeotropic distillation process, adsorption, column chromatography, simple vacuum distillation and microfiltration, combined or not.

Recovered monomers and/or oligomers may be reused for instance to synthesize plastics. In some embodiments, the recovered monomers and/or oligomers are used to synthesize polyesters, including but not limited to PLA and PET. Advantageously, polyesters of same nature are repolymerized. In some embodiments, the recovered monomers and/or oligomers are mixed with other monomers and/or oligomers, for example to synthesize new copolymers. Alternatively, the recovered monomers may be used as chemical intermediates to produce other chemical compounds of interest.

In certain embodiments, the method is employed for surface hydrolysis or surface functionalization of a plastic-containing material, comprising exposing a plastic-containing material to an enzyme described herein, or corresponding recombinant cell or extract thereof, or composition. In some embodiments, the plastic material consists of a polyester. The method is particularly useful for increasing hydrophilicity, or water absorbency, of a polyester material. Such increased hydrophilicity may have particular interest in textiles production, electronics and biomedical applications.

### Embodiments

Notwithstanding the appended claims, the present disclosure is also defined by the following embodiments.
1. A method of assessing an enzyme for plastic-degrading activity, the method comprising:
   contacting a plastic with the enzyme under conditions suitable for plastic-degrading enzyme activity; and
   assessing for degradation of the plastic by the enzyme.
2. The method according to embodiment 1, wherein the plastic is a plastic substrate.
3. The method according to embodiment 2, wherein the plastic substrate is a homogenous plastic substrate.
4. The method according to embodiment 2 or embodiment 3, wherein the plastic substrate comprises a solid film of the plastic.
5. The method according to embodiment 2 or embodiment 3, wherein the plastic substrate comprises a solid sheet of the plastic.
6. The method according to embodiment 2, wherein the plastic substrate is a fiber, fabric, textile, or combination thereof comprising the plastic.
7. The method according to any one of embodiments 2 to 6, wherein the contacting comprises contacting the plastic with a liquid comprising cells that secrete and/or display the enzyme.
8. The method according to any one of embodiments 2 to 6, wherein the contacting comprises contacting the plastic with a liquid comprising the enzyme in purified form.
9. The method according to embodiment 1, wherein the contacting comprises suspending the enzyme in a solid or semi-solid medium comprising the plastic.
10. The method according to embodiment 9, wherein the solid or semi-solid medium is a solid or semi-solid agar medium.
11. The method according to embodiment 9 or embodiment 10, wherein the contacting comprises growing cells that secrete and/or display the enzyme in the solid or semi-solid medium, wherein the solid or semi-solid medium is a growth medium for the cells.
12. The method according to embodiment 11, wherein the cells are bacterial cells.
13. The method according to embodiment 12, wherein the cells are Escherichia coli cells.
14. The method according to embodiment 11, wherein the cells are fungal cells.
15. The method according to embodiment 14, wherein the fungal cells are yeast cells.
16. The method according to embodiment 15, wherein the yeast cells are Saccharomyces cerevisiae cells or Pichia pastoris cells.
17. The method according to any one of embodiments 11 to 16, wherein the assessing comprises assessing for the presence, size, or both of halos at the locations of cell colonies in the solid or semi-solid growth medium.
18. The method according to embodiment 1, wherein:
   the plastic comprises plastic nanoparticles;
   the contacting comprises providing the enzyme in a suspension comprising the plastic nanoparticles; and
   the assessing comprises assessing for degradation of the plastic nanoparticles.
19. The method according to embodiment 18, wherein the contacting comprises providing a cell lysate comprising the enzyme in the suspension comprising the plastic nanoparticles.
20. The method according to embodiment 18, wherein the contacting comprises providing a supernatant or media component of a cell culture comprising the enzyme in the suspension comprising the plastic nanoparticles.
21. The method according to embodiment 18, wherein the contacting comprises providing cells that secrete or display the enzyme in the suspension comprising the plastic nanoparticles.
22. The method according to any one of embodiments 19 to 21, wherein the cell lysate, supernatant, media component, or cells are from cells or progeny thereof obtained from a cell colony identified as producing a halo of a threshold size according to the method of embodiment 17.
23. The method according to any one of embodiments 18 to 22, wherein the assessing is by spectroscopy.
24. The method according to embodiment 23, wherein the assessing is by determining optical density.
25. The method according to any one of embodiments 18 to 22, wherein the assessing comprises detecting a compound that emits a detectable signal upon release from a degrading plastic nanoparticle.
26. The method according to embodiment 25, wherein the compound is a compound that fluoresces upon release from the degrading plastic nanoparticle.
27. The method according to any one of embodiments 18 to 22, wherein the assessing is by liquid chromatography, gas chromatography, liquid chromatography-mass spectrometry (LC-MS), or gas chromatography-mass spectrometry (GC-MS).
28. The method according to any one of embodiments 18 to 27, wherein the suspension comprising the plastic nanoparticles is present in an isolated well having an open or closed end.
29. The method according to embodiment 28, wherein the isolated well is present in a micro-capillary array or multi-well plate.
30. The method according to any one of embodiments 1 to 29, wherein the plastic comprises, consists essentially of, or consists of polylactic acid (PLA).
31. The method according to any one of embodiments 1 to 29, wherein the plastic comprises, consists essentially of, or consists of polyethylene terephthalate (PET).
32. The method according to any one of embodiments 1 to 29, wherein the plastic comprises, consists essentially of, or consists of polystyrene.
33. The method according to any one of embodiments 1 to 32, wherein the enzyme is a variant of a parental enzyme.
34. The method according to embodiment 33, wherein the parental enzyme is known to have plastic-degrading activity.
35. The method according to embodiment 34, wherein the assessing comprises assessing for whether the variant enzyme exhibits enhanced plastic-degrading activity as compared to the parental enzyme.
36. The method according to any one of embodiments 33 to 35, wherein the parental enzyme is a cutinase or cutinase-like enzyme.
37. The method according to embodiment 36, wherein the cutinase or cutinase-like enzyme is a cutinase-like enzyme from Cryptococcus sp. (CLE1).
38. The method according to embodiment 36, wherein the cutinase or cutinase-like enzyme is a cutinase from Humicola insolens (HiC).
39. A polylactic acid (PLA)-degrading enzyme comprising an amino acid sequence having 70% or greater, 75% or greater, 80% or greater, 85% or greater, 90% or greater, 95% or greater, 96% or greater, 97% or greater, 98% or greater, 99% or greater, or 100% identity to the amino acid sequence set forth in any one of SEQ ID NOS:3, 5, 24-25, or a PLA-degrading fragment thereof.
40. The PLA-degrading enzyme of embodiment 39, wherein the PLA-degrading enzyme comprises an amino acid substitution at A40, Y94, Q111, Q112, A118, T148, 1191, Q202, or any combination thereof, wherein numbering is as in SEQ ID NO:3.
41. The PLA-degrading enzyme of embodiment 40, wherein the PLA-degrading enzyme comprises the amino acid substitution A40V, Y94F, Q111K, Q112R, Al18P, T1481, 1191V, Q202E, or any combination thereof.
42. The PLA-degrading enzyme of embodiment 41, wherein the PLA-degrading enzyme comprises the amino acid substitutions A40V, Q112R and 1191V.
43. A nucleic acid encoding the PLA-degrading enzyme of any one of embodiments 39 to 42.
44. A cell comprising the nucleic acid of embodiment 43, optionally wherein the cell is a yeast cell, such as, Saccharomyces species, e.g., Saccharomyces cerevisiae or Pichia species, e.g., Pichia pastoris.
45. The cell of embodiment 44, wherein the nucleic acid is present in an expression vector.
46. A method of producing the PLA-degrading enzyme of any one of embodiments 39 to 42, comprising culturing the cell of embodiment 45 under conditions suitable for expression of the PLA-degrading enzyme, wherein the PLA-degrading enzyme is produced.
47. A method of degrading PLA, the method comprising contacting PLA with the PLA-degrading enzyme of any one of embodiments 39 to 42 under conditions suitable for degradation of the PLA by the PLA-degrading enzyme.
48. A polyethylene terephthalate (PET)-degrading enzyme comprising an amino acid sequence having 70% or greater, 75% or greater, 80% or greater, 85% or greater, 90% or greater, 95% or greater, 96% or greater, 97% or greater, 98% or greater, 99% or greater, or 100% identity to the amino acid sequence set forth in any one of SEQ ID NOS:4, 6-23, or a PET-degrading fragment thereof.
49. The PET-degrading enzyme of embodiment 48, wherein the PET-degrading enzyme comprises an amino acid substitution at Q1, G3, A4, S11, N15, T29, T37, E47, T164, T166, or any combination thereof, wherein numbering is as in SEQ ID NO:4.
50. The PET-degrading enzyme of embodiment 49, wherein the PET-degrading enzyme comprises the amino acid substitution Q1A, Q1C, Q1 D, Q1E, Q1F, Q1G, Q1H, Q1K, Q1L, Q1M, Q1N, Q1P, Q1R, Q1S, Q1T, Q1V, Q1Y, G3A, G3C, G3D, G3E, G3F, G3H, G31, G3L, G3N, G3Q, G3R, G3S, G3T, G3V, G3Y, A4V, S11N, N15D, T29K, T37A, T371, T37L, T37M, T37N, T37P, T37S, T37V, E47A, E47C, E47F, E47G, E47H, E471, E47L, E47M, E47N, E47Q, E47R, E47S, E47T, E47V, E47Y, T164S, or any combination thereof at different positions.
51. The PET-degrading enzyme of embodiment 50, wherein the PET-degrading enzyme comprises the amino acid substitutions Q1R and T29K.
52. The PET-degrading enzyme of embodiment 51, wherein the PET-degrading enzyme comprises the amino acid substitutions Q1R, T29K and E47Q.
53. The PET-degrading enzyme of embodiment 52, wherein the PET-degrading enzyme comprises the amino acid substitutions Q1R, G3S, T29K and E47Q.
54. The PET-degrading enzyme of any one of embodiments 50 to 52, wherein the PET-degrading enzyme comprises the amino acid substitution E47R.
55. The PET-degrading enzyme of embodiment 50, wherein the PET-degrading enzyme comprises the amino acid substitutions Q11, T37L, and E47R.
56. The PET-degrading enzyme of embodiment 50, wherein the PET-degrading enzyme comprises the amino acid substitutions 11, G3S, T371, and E47R.
57. The PET-degrading enzyme of embodiment 50, wherein the PET-degrading enzyme comprises the amino acid substitutions Q1R, G3T, T37L, and E47R.
58. A nucleic acid encoding the PET-degrading enzyme of any one of embodiments 48 to 57.
59. A cell comprising the nucleic acid of embodiment 58, optionally wherein the cell is a yeast cell, such as, Saccharomyces species, e.g., Saccharomyces cerevisiae, e.g., Saccharomyces cerevisiae or Pichia species, e.g., Pichia pastoris.
60. The cell of embodiment 59, wherein the nucleic acid is present in an expression vector.
61. A method of producing the PET-degrading enzyme of any one of embodiments 48 to 57, comprising culturing the cell of embodiment 60 under conditions suitable for expression of the PET-degrading enzyme, wherein the PET-degrading enzyme is produced.
62. A method of degrading PET, the method comprising contacting PET with the PET-degrading enzyme of any one of embodiments 48 to 57 under conditions suitable for degradation of the PET by the PET-degrading enzyme.

The following examples are offered by way of illustration and not by way of limitation.

### EXPERIMENTAL

### Example 1—Identification and Application of Enzymes and Variants Thereof Enhanced in Degradation of Polylactic Acid (PLA)

A mutagenized library was prepared using DNA encoding the wildtype, mature protein sequence of cutinase-like enzyme from Cryptococcus sp. (SEQ ID NO:1, hereafter referred to as CLE1) codon-optimized for expression in Saccharomyces cerevisiae. Mutagenesis was performed using a GeneMorph II kit (Agilent Technologies) following manufacturer's directions. A ura3-complementing, galactose-inducible expression vector was linearized by restriction digest and gel purified. Vector and mutagenized insert DNA were precipitated using sodium acetate and isopropanol and combined for transformation into a ura3-knockout strain of S. cerevisiae by the lithium acetate method and electroporation. Transformed cultures were passaged three times every 24 hours into uracil-deficient growth media to select for transformants. Enzyme expression and extracellular localization were achieved by addition of galactose. PLA nanoparticles were embedded in solidified growth medium. The passaged culture was spread on PLA-containing growth plates and incubated at 30 C to develop halos. 360 colonies having halos were identified and picked into uracil-deficient liquid in a 2-ml deep-96-well plate and grown overnight with temperature and humidity control and orbital shaking. Enzyme expression and extracellular localization were achieved by galactose addition. Plates were centrifuged to separate cells from enzyme-containing culture supernatant. The culture supernatant was combined with a PLA nanoparticle suspension. Enzyme activity was monitored via absorbance at 600 nm. Variants corresponding to wells with the highest absorbance decrease over time were selected and recombined via PCR and Gibson assembly to produce all possible combinations of identified mutations. These combinations were subjected to the same screening procedure described above. Mutation combinations with the highest activity were used as the starting point for the next round of mutagenesis and screening.

After the first round of CLE1 screening, the single mutants A40V, Q111K, and Q112R were each identified as having enhanced PLA degradation activity. Validation of mutation combinations yielded A40V+Q112R as having the highest enzyme activity improvement. This double mutant was used as the starting point of a second round of screening, which yielded additional mutations 1191V, A118P, Y94F, and T1481+Q202E as having enhanced PLA degradation activity relative to the starting point. Validation revealed A40V+Q112R+1191V having the highest enzyme activity. This triple mutant was expressed in S. cerevisiae and purified using ammonium sulfate precipitation and size exclusion chromatography to homogeneity.

In one application, the purified A40V+Q112R+1191V CLE1 triple mutant was tested for degradation of a section of a 3 oz World Centric PLA-based cold cup (product code CP-CS-3) in 0.1 M potassium phosphate buffer (pH 8.0) at room temperature for 7 days under saturating relative humidity. PLA degradation was evidenced by a decrease in pH to below 5 and increased opacity in the enzyme-treated area. Neither effect was observed in a control experiment without enzyme.

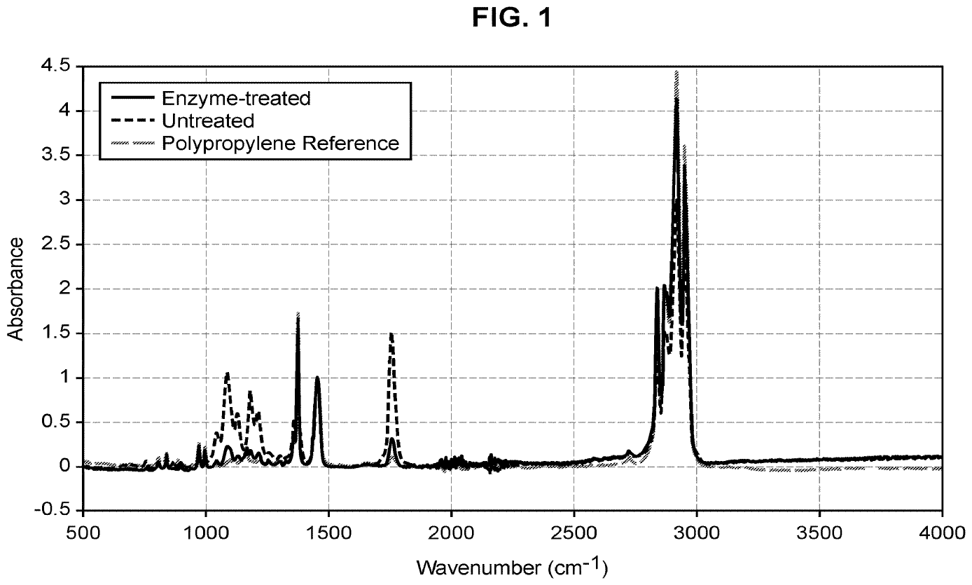

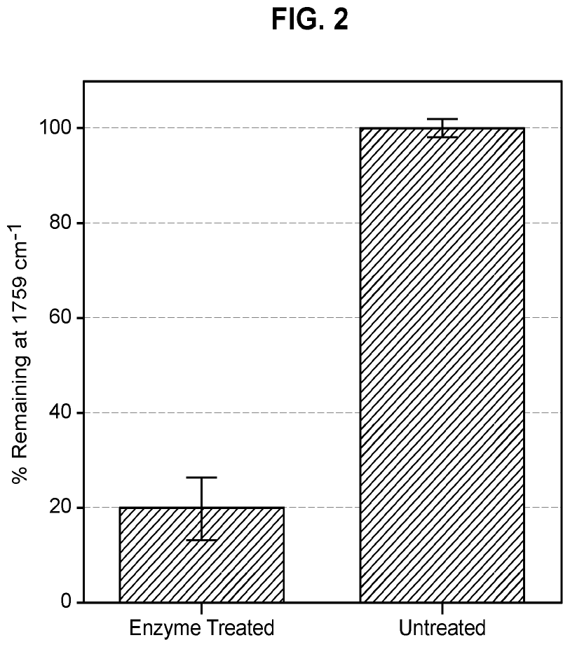

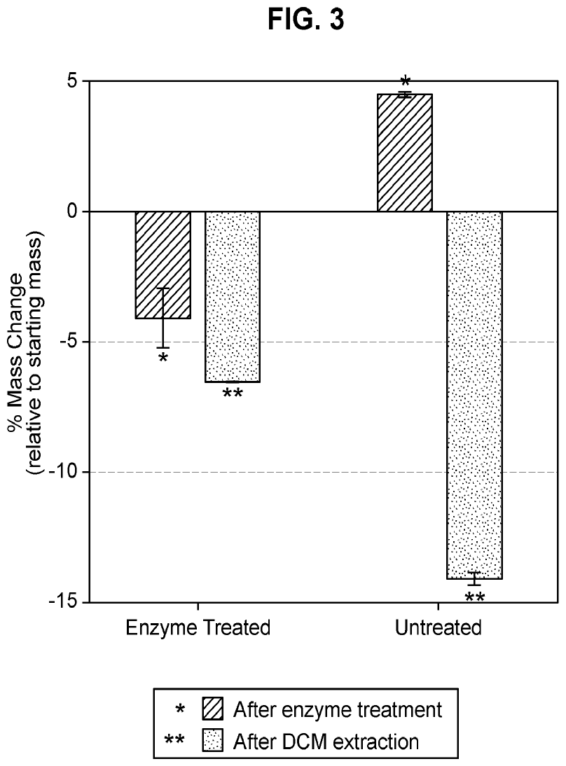

In another application, the purified A40V+Q112R+1191V CLE1 triple mutant was tested for degradation of a non-woven fabric containing 15% PLA fibers and 85% polypropylene fibers by weight. Fabric pieces weighing between 160 mg and 172 mg were cut from fabric and used as is without other processing. Fabric pieces were combined with 0.5 M potassium phosphate buffer (pH 8.0) containing 0.1% w/v sodium dodecyl sulfate at 30 C with shaking at 280 rpm in 20-ml sealed scintillation vials. Solids loading was 10% w/v fabric and enzyme concentration was 0.15 mg/ml. After 9 days, the liquid phase was removed from the vials and the remaining solids washed extensively with distilled water before drying at 50 C. Characterization by Fourier transform infrared spectroscopy indicated an average 80% reduction in PLA content relative to untreated fabric and a polypropylene-only control, calculated by the reduction of the absorbance peak at 1759 cm⁻¹ corresponding to C═O stretching relative to the control samples (FIG. 1 and FIG. 2). After solvent extraction of residual PLA using dichloromethane, gravimetric analysis of the fabric pieces indicated an average 54% reduction in PLA content relative to untreated fabric (FIG. 3).

### Example 2—Identification and Application of Enzymes and Variants Thereof Enhanced in Degradation of Polyethylene Terephthalate (PET) Plastics

A mutagenized library was prepared using DNA encoding the wildtype, mature protein sequence of cutinase from Humicola insolens (SEQ ID NO:2, hereafter referred to as HiC) codon-optimized for expression in Saccharomyces cerevisiae. Mutagenesis was performed using a GeneMorph II kit (Agilent Technologies) following manufacturer's directions. A ura3-complementing, galactose-inducible expression vector was linearized by restriction digest and gel purified. Vector and mutagenized insert DNA were precipitated using sodium acetate and isopropanol and combined for transformation into a ura3-knockout strain of S. cerevisiae by the lithium acetate method and electroporation. Transformed cultures were passaged three times every 24 hours into uracil-deficient growth media to select for transformants. Enzyme expression and extracellular localization were achieved by addition of galactose. Solid growth medium was prepared using agar, sodium dodecyl sulfate, and bistoluoyl bis(2-hydroxyethyl)terephthalate. The passaged culture was spread on this growth medium and incubated at 30 C to develop halos. 360 colonies having halos were identified and picked into uracil-deficient liquid in a 2-ml deep-96-well plate and grown overnight with temperature and humidity control and orbital shaking. Enzyme expression and extracellular localization were achieved by galactose addition. Plates were centrifuged to separate cells from enzyme-containing culture supernatant. The culture supernatant was combined with a PET nanoparticle suspension and incubated at 60 C. Enzyme activity was monitored via fluorescence. Variants corresponding to wells with the highest activity were selected and recombined via PCR and Gibson assembly to produce all possible combinations of identified mutations. These combinations were subjected to the same screening procedure described above. Mutation combinations with the highest activity were used as the starting point for the next round of mutagenesis and screening.

After one round of screening, the single mutants A4V, T164S, T29K, G3S, E47Q, Q1R, S11 N, and N15D were each identified as having enhanced PET degradation activity at 68 C. The following combinations of mutations were assembled and assayed for PET degradation activity as above: Q1R+G3S, Q1R+T29K, Q1R+E47Q, G3S+T29K, T29K+E47Q, Q1R+G3S+T29K, Q1R+G3S+E47Q, Q1R+T29K+E47Q, G3S+T29K+E47Q, and Q1R+G3S+T29K+E47Q.

TABLE 1
Specific PET-degrading activity of HiC variants
relative to wildtype HiC (SEQ ID NO: 4)

| Variant | PET-Degrading Activity |
|---|---|
| HiC[Q1R + G3S + T29K + E47Q] | 149% |
| HiC[Q1R + T29K + E47Q] | 144% |
| HiC[Q1R + G3S + T29K] | 140% |
| HiC[Q1R + T29K] | 140% |
| HiC[G3S + T29K + E47Q] | 134% |
| HiC[T29K + E47Q] | 134% |
| HiC[G3S + T29K] | 127% |
| HiC[T29K] | 124% |
| HiC[Q1R + G3S + E47Q] | 122% |
| HiC[Q1R + E47Q] | 119% |
| HiC[G3S + E47Q] | 117% |
| HiC[A4V] | 116% |
| HiC[Q1R] | 113% |
| HiC[E47Q] | 113% |
| HiC[Q1R + G3S] | 110% |
| HiC[T164S] | 108% |
| HiC[S11N] | 105% |
| HiC[N15D] | 104% |
| HiC[G3S] | 103% |

In a separate round of screening, site-saturation mutagenesis was performed on the four residues Q1, G3, T37, and E47 of the HiC parent enzyme in combination. Variants were expressed and analyzed using PET nanoparticles as described above. Combinations of mutations listed below were identified as having enhanced PET degradation activity at 68 C relative to wildtype HiC.

$$Q 1 P + G 3 Q + T 37 I + E 47 R$$

$$Q 1 R + G 3 T + T 37 L + E 47 R$$

$$Q 1 G + G 3 R + T 37 M + E 47 R$$

$$Q 1 F + G 3 N + T 37 L + E 47 A$$

$$Q 1 S + G 3 R + T 37 I + E 47 S$$

$$Q 1 R + G 3 F + T 37 L + E 47 L$$

$$Q 1 M + G 3 F + T 37 A + E 47 R$$

$$Q 1 N + G 3 L + T 37 N + E 47 R$$

$$Q 1 H + G 3 L + T 37 L + E 47 S$$

$$Q 1 G + G 3 L + T 37 L + E 47 Y$$

$$Q 1 T + E 47 R$$

$$Q 1 N + G 3 Y + T 37 L + E 47 S$$

$$Q 1 E + G 3 N + T 37 V + E 47 R$$

$$Q 1 L + G 3 R + T 37 N + E 47 R$$

$$Q 1 S + T 37 V + E 47 C$$

$$Q 1 V + G 3 Y + T 37 I + E 47 V$$

$$Q 1 Y + G 3 N + T 37 L + E 47 S$$

$$Q 1 P + G 3 L + T 37 I + E 47 M$$

$$Q 1 H + G 3 H + T 37 S + E 47 S$$

$$Q 1 M + G 3 D + T 37 S + E 47 R$$

$$Q 1 K + G 3 Y + T 37 L + E 47 F$$

$$Q 1 G + G 3 R + E 47 I$$

$$Q 1 S + G 3 A + T 37 L + E 47 Q$$

$$Q 1 S + G 3 I + T 37 I + E 47 L$$

$$Q 1 H + G 3 V + T 37 L + E 47 V$$

$$Q 1 A + G 3 L + T 37 L + E 47 G$$

$$Q 1 H + G 3 V + T 37 S + E 47 Q$$

$$Q 1 E + G 3 E + T 37 V + E 47 R$$

$$T 37 S + E 47 M$$

$$Q 1 K + G 3 Q + T 37 S = E 47 H$$

$$Q 1 K + G 3 V + T 37 A + E 47 A$$

$$Q 1 C + G 3 N + E 47 Y$$

$$Q 1 H + G 3 A + T 37 A + E 47 S$$

$$Q 1 G + G 3 T + E 47 V$$

$$Q 1 L + G 3 S + T 37 M + E 47 V$$

$$Q 1 R + G 3 C + T 37 N + E 47 S$$

$$Q 1 G + G 3 L + T 37 A + E 47 T$$

$$Q 1 S + G 3 N + T 37 L + E 47 G$$

$$Q 1 A + G 3 Y + T 37 S + E 47 V$$

$$Q 1 S + T 37 S + E 47 G$$

In subsequent validation of this round of screening, the mutation E47R was found to contribute significantly to improved degradation activity of PET nanoparticles at 68 C, and the combinations Q11+T37L+E47R, Q1 I+G3S+T371+E47R, and Q1R+G3T+T37L+E47R were found to be significantly improved relative to wildtype as well as the Q1R+G3S+T29K+E47Q mutant described above. These activities are reported in Table 2.

TABLE 2
Specific PET-degrading activity of HiC variants
relative to wildtype HiC (SEQ ID NO: 4)

| Variant | PET-Degrading Activity |
|---|---|
| HiC[Q1I + T37L + E47R] | 291% |
| HiC[Q1R + G3T + T37L + E47R] | 266% |
| HiC[Q1I + G3S + T37I + E47R] | 259% |
| HiC[Q1R + T29K + E47Q] | 203% |
| HiC[Q1R + G3S + T29K + E47Q] | 201% |
| HiC[Q1R + T29K] | 168% |
| HiC[T29K] | 153% |

Wildtype HiC and its mutants T29K, Q1R+T29K, Q1R+T29K+E47Q, Q1R+G3S+T29K+E47Q were cloned into a pET24a E. coli expression vector containing a C-terminal His6 purification tag. E. coli harboring these expression vectors were cultured in 1 liter LBE505 media and induced with 0.5 mM isopropyl β-D-1-thiogalactopyranoside (IPTG) at OD-600 1.8 and cultured at 20 C for 20 hours. Bacterial cells were lysed by freeze-thaw cycling and sonication and expressed enzyme purified out of the lysate supernatant using cobalt affinity resin followed by ion exchange chromatography. Purified protein concentrations were measured by absorbance at 280 nm. Purified mutants were tested for activity against commercially available PET fiber (Goodfellow ES305710/1, 3.3 tex, 0.014 mm filament diameter, 15 filaments). Reactions involved 24.75 mg/ml fiber and 0.1 mg/ml purified enzyme in 100 mM potassium phosphate and 100 mM sodium chloride, pH 8.0, with incubation at 60 C and agitation at 460 rpm for 50 hours. Enzyme activity was assessed by quantifying terephthalic acid and 2-monohydroxyethylterephthalate production by liquid chromatography mass spectrometry. The evolved mutants all displayed improved product formation relative to wildtype HiC as reported in Table 3.

TABLE 3
Specific activity of HiC variants on PET fiber
relative to wildtype HiC (SEQ ID NO: 4)

| Variant | PET-Degrading Activity |
|---|---|
| HiC[Q1R + G3S + T29K + E47Q] | 214% |
| HiC[Q1R + T29K + E47Q] | 178% |
| HiC[Q1R + T29K] | 120% |
| HiC[T29K] | 107% |

NUCLEOTIDE SEQUENCES
SEQ ID NO: 1: Cutinase-like enzyme from Cryptococcus sp.
GCGCCGACTCCTGAAAGCGCCGAGGCTCATGAGCTGGAAGCTAGGGCCACTTCATCCGC
TTGTCCACAATACGTTTTGATAAACACCAGGGGCACAGGAGAACCACAGGGGCAATCAGC
CGGCTTCAGGACCATGAATAGTCAGATTACCGCAGCATTGTCAGGGGGCACCATATATAAT
ACAGTTTATACCGOGGATTTCTCACAGAACAGTGCCGCGGGGACAGCAGACATAATTCGT
AGGATCAATAGCGGTTTAGCAGCGAATCCCAATGTCTGTTATATTCTACAGGGGTACTCAC
AGGGAGCGGCCGCGACCGTCGTTGCGTTACAACAACTAGGCACCTCCGGGGCTGCATTC
AACGCCGTTAAAGGAGTCTTCCTTATCGGGAACCCGGACCACAAATCTGGGCTTACGTGC
AATGTCGATAGCAATGGTGGGACCACCACCCGTAATGTCAATGGCTTGTCAGTCGCTTAC
CAAGGCTCAGTGCCTTCAGGTTGGGTCTCAAAGACTTTGGACGTATGTGCCTATGGGGAT
GGCGTGTGCGATACTGCACATGGGTTTGGTATCAATGCCCAACATTTATCTTATCCTTCAG
ACCAGGGAGTCCAGACAATGGGCTACAAATTTGCGGTCAACAAATTGGGGGGTAGTGCT
SEQ ID NO: 2: Cutinase from Humicola insolens
CAACTAGGTGCTATCGAAAATGGGTTAGAGAGCGGCTCCGCAAATGCTTGTCCCGATGCT
ATATTGATCTTTGCAAGGGGAaCAACGGAACCCGGTAACATGGGAATTttAGTTGGCCCTGC
ACTTGCGAATGGGTTAGAATCACACATTCGTAATATTTGGATACAAGGCGTAGGGGGACCC
TACGATGCTGCTCTAGCCACAAACTTCCTGCCTCGTGGCACCAGTCAAGCCAACATTGAC
GAAGGAAAGAGATTGTTTGCGCTTGCTAACCAGAAATGTCCCAATACCCCCGTTGTTGCAG
GTGGTTATTCACAGGGCGCAGCACTTATTGCTGCGGCGGTCTCCGAGTTATCTGGGGCAG
TGAAGGAGCAAGTTAAAGGCGTGaCTTTGTTTGGATATACCCAGAATTTACAAAATAtGGGC
GGCATCCCAAATTACCCTCGTGAACGTACAAAGGTCTTCTGCAACGCCGGCGATGCCGTG
TGCACTGGCACGCTGATAATTACCCCTGCACACCTATCATACACAATAGAGGCTAGAGGC
GAGGCGGCACGTTTCTTGAGGGACAGAATACGTGCA
AMINO ACID SEQUENCES
SEQ ID NO: 3: Cutinase-like enzyme from Cryptococcus sp. (CLE1)
APTPESAEAHELEARATSSACPQYVLINTRGTGEPQGQSAGFRTMNSQITAALSGGTIYNTVY
TADFSQNSAAGTADIIRRINSGLAANPNVCYILQGYSQGAAATVVALQQLGTSGAAFNAVKGVF
LIGNPDHKSGLTCNVDSNGGTTTRNVNGLSVAYQGSVPSGWVSKTLDVCAYGDGVCDTAHG
FGINAQHLSYPSDQGVQTMGYKFAVNKLGGSA
SEQ ID NO: 4: Cutinase from Humicola insolens (HiC)
QLGAIENGLESGSANACPDAILIFARGSTEPGNMGITVGPALANGLESHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCTGTLIITPAHLSYTIEARGEAARFLRDRI
RA
SEQ ID NO: 5: Cutinase from Saitozyma flava
MLFSAIALAALSAASMSCAAPTPESAAGHELEARATSSACPQYVLINTRGTGEPQGQSAGFRT
MNSQITAALSGGTIYNTVYTADFSQNSAAGTADIIRRINSGLASNPNVCYILQGYSQGAAATVVA
LQQLGTSGAAFNAVKGVFLIGNPDHKAGLTCNVDSNGGTTTRNVNGLSVAYQGSIPSGWVSK
TLDVCAYGDGVCDTTHGYGINAPHLSYPNDQGVQSMGYKFAVNKLGGSA
SEQ ID NO: 6: HiCa
ILGAIENGLESGSANACPDAILIFARGSTEPGNMGILVGPALANGLRSHIRNIWIQGVGGPYDAA
LATNFLPRGTSQANIDEGKRLFALANQKCPNTPVVAGGYSQGAALIAAAVSELSGAVKEQVKG
VALFGYTQNLQNRGGIPNYPRERTKVFCNVGDAVCTGTLIITPAHLSYTIEARGEAARFLRDRIR
A
SEQ ID NO: 7: HiCb
ILSAIENGLESGSANACPDAILIFARGSTEPGNMGIIVGPALANGLRSHIRNIWIQGVGGPYDAAL
ATNFLPRGTSQANIDEGKRLFALANQKCPNTPVVAGGYSQGAALIAAAVSELSGAVKEQVKGV
ALFGYTQNLQNRGGIPNYPRERTKVFCNVGDAVCTGTLIITPAHLSYTIEARGEAARFLRDRIRA
SEQ ID NO: 8: HiCc
RLTAIENGLESGSANACPDAILIFARGSTEPGNMGILVGPALANGLRSHIRNIWIQGVGGPYDAA
LATNFLPRGTSQANIDEGKRLFALANQKCPNTPVVAGGYSQGAALIAAAVSELSGAVKEQVKG
VALFGYTQNLQNRGGIPNYPRERTKVFCNVGDAVCTGTLIITPAHLSYTIEARGEAARFLRDRIR
A
SEQ ID NO: 9: HiCd
RLSAIENGLESGSANACPDAILIFARGSKEPGNMGITVGPALANGLQSHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCTGTLIITPAHLSYTIEARGEAARFLRDRI
RA
SEQ ID NO: 10: HiCe
QLGAIENGLENGSADACPDAILIFARGSTEPGNMGITVGPALANGLESHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCSGTLIITPAHLSYTIEARGEAARFLRDRI
RA
SEQ ID NO: 11: HiCf
QLGVIENGLESGSADACPDAILIFARGSTEPGNMGITVGPALANGLESHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCSGTLIITPAHLSYTIEARGEAARFLRDRI
RA
SEQ ID NO: 12: HiCg
QLGVIENGLENGSANACPDAILIFARGSTEPGNMGITVGPALANGLESHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCSGTLIITPAHLSYTIEARGEAARFLRDRI
RA
SEQ ID NO: 13: HiCh
QLGVIENGLENGSADACPDAILIFARGSTEPGNMGITVGPALANGLESHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCTGTLIITPAHLSYTIEARGEAARFLRDRI
RA
SEQ ID NO: 14: HiCi
QLGVIENGLENGSANACPDAILIFARGSTEPGNMGITVGPALANGLESHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCTGTLIITPAHLSYTIEARGEAARFLRDRI
RA
SEQ ID NO: 15: HiCj
QLGVIENGLESGSADACPDAILIFARGSTEPGNMGITVGPALANGLESHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCTGTLIITPAHLSYTIEARGEAARFLRDRI
RA
SEQ ID NO: 16: HiCk
QLGVIENGLESGSANACPDAILIFARGSTEPGNMGITVGPALANGLESHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCSGTLIITPAHLSYTIEARGEAARFLRDRI
RA
SEQ ID NO: 17: HiCl
QLGAIENGLENGSADACPDAILIFARGSTEPGNMGITVGPALANGLESHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCTGTLIITPAHLSYTIEARGEAARFLRDRI
RA
SEQ ID NO: 18: HiCm
QLGAIENGLENGSANACPDAILIFARGSTEPGNMGITVGPALANGLESHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCSGTLIITPAHLSYTIEARGEAARFLRDRI
RA
SEQ ID NO: 19: HiCn
QLGAIENGLESGSADACPDAILIFARGSTEPGNMGITVGPALANGLESHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCSGTLIITPAHLSYTIEARGEAARFLRDRI
RA
SEQ ID NO: 20: HiCo
QLGVIENGLESGSANACPDAILIFARGSTEPGNMGITVGPALANGLESHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCTGTLIITPAHLSYTIEARGEAARFLRDRI
RA
SEQ ID NO: 21: HiCp
QLGAIENGLENGSANACPDAILIFARGSTEPGNMGITVGPALANGLESHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCTGTLIITPAHLSYTIEARGEAARFLRDRI
RA
SEQ ID NO: 22: HiCq
QLGAIENGLESGSADACPDAILIFARGSTEPGNMGITVGPALANGLESHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCTGTLIITPAHLSYTIEARGEAARFLRDRI
RA
SEQ ID NO: 23: HiCr
QLGAIENGLESGSANACPDAILIFARGSTEPGNMGITVGPALANGLESHIRNIWIQGVGGPYDA
ALATNFLPRGTSQANIDEGKRLFALANQKCPNTPVVAGGYSQGAALIAAAVSELSGAVKEQVK
GVALFGYTQNLQNRGGIPNYPRERTKVFCNVGDAVCSGTLIITPAHLSYTIEARGEAARFLRDRI
RASEQ
ID NO: 24: CLE1A
APTPESAEAHELEARATSSACPQYVLINTRGTGEPQGQSAGFRTMNSQITAALSGGTIYNTVY
TADFSQNSAAGTADIIRRINSGLAANPNVCFILQGYSQGAAATVVALKQLGTSGPAFNAVKGVF
LIGNPDHKSGLTCNVDSNGGITTRNVNGLSVAYQGSVPSGWVSKTLDVCAYGDGVCDTAHGF
GINAQHLSYPSDEGVQTMGYKFAVNKLGGSA
SEQ ID NO: 25: CLE1B
APTPESAEAHELEARATSSACPQYVLINTRGTGEPQGQSAGFRTMNSQITAALSGGTIYNTVY
TADFSQNSAAGTADIIRRINSGLAANPNVCFILQGYSQGAAATVVALKQLGTSGPAFNAVKGVF
LIGNPDHKSGLTCNVDSNGGITTRNVNGLSVAYQGSVPSGWVSKTLDVCAYGDGVCDTAHGF
GINAQHLSYPSDQGVQTMGYKFAVNKLGGSA
SEQ ID NO: 26: CLE1C
APTPESAEAHELEARATSSACPQYVLINTRGTGEPQGQSAGFRTMNSQITAALSGGTIYNTVY
TADFSQNSAAGTADIIRRINSGLAANPNVCYILQGYSQGAAATVVALKQLGTSGPAFNAVKGVF
LIGNPDHKSGLTCNVDSNGGITTRNVNGLSVAYQGSVPSGWVSKTLDVCAYGDGVCDTAHGF
GINAQHLSYPSDEGVQTMGYKFAVNKLGGSA
SEQ ID NO: 27: CLE1D
APTPESAEAHELEARATSSACPQYVLINTRGTGEPQGQSAGFRTMNSQITAALSGGTIYNTVY
TADFSQNSAAGTADIIRRINSGLAANPNVCFILQGYSQGAAATVVALQQLGTSGPAFNAVKGVF
LIGNPDHKSGLTCNVDSNGGITTRNVNGLSVAYQGSVPSGWVSKTLDVCAYGDGVCDTAHGF
GINAQHLSYPSDEGVQTMGYKFAVNKLGGSA
SEQ ID NO: 28: CLE1E
APTPESAEAHELEARATSSACPQYVLINTRGTGEPQGQSAGFRTMNSQITAALSGGTIYNTVY
TADFSQNSAAGTADIIRRINSGLAANPNVCFILQGYSQGAAATVVALKQLGTSGAAFNAVKGVF
LIGNPDHKSGLTCNVDSNGGITTRNVNGLSVAYQGSVPSGWVSKTLDVCAYGDGVCDTAHGF
GINAQHLSYPSDEGVQTMGYKFAVNKLGGSA
SEQ ID NO: 29: CLE1F
APTPESAEAHELEARATSSACPQYVLINTRGTGEPQGQSAGFRTMNSQITAALSGGTIYNTVY
TADFSQNSAAGTADIIRRINSGLAANPNVCFILQGYSQGAAATVVALKQLGTSGPAFNAVKGVF
LIGNPDHKSGLTCNVDSNGGTTTRNVNGLSVAYQGSVPSGWVSKTLDVCAYGDGVCDTAHG
FGINAQHLSYPSDEGVQTMGYKFAVNKLGGSA
SEQ ID NO: 30: CLE1G
APTPESAEAHELEARATSSACPQYVLINTRGTGEPQGQSAGFRTMNSQITAALSGGTIYNTVY
TADFSQNSAAGTADIIRRINSGLAANPNVCFILQGYSQGAAATVVALQQLGTSGAAFNAVKGVF
LIGNPDHKSGLTCNVDSNGGTTTRNVNGLSVAYQGSVPSGWVSKTLDVCAYGDGVCDTAHG
FGINAQHLSYPSDQGVQTMGYKFAVNKLGGSA
SEQ ID NO: 31: CLE1H
APTPESAEAHELEARATSSACPQYVLINTRGTGEPQGQSAGFRTMNSQITAALSGGTIYNTVY
TADFSQNSAAGTADIIRRINSGLAANPNVCYILQGYSQGAAATVVALKQLGTSGAAFNAVKGVF
LIGNPDHKSGLTCNVDSNGGTTTRNVNGLSVAYQGSVPSGWVSKTLDVCAYGDGVCDTAHG
FGINAQHLSYPSDQGVQTMGYKFAVNKLGGSA
SEQ ID NO: 32: CLE1I
APTPESAEAHELEARATSSACPQYVLINTRGTGEPQGQSAGFRTMNSQITAALSGGTIYNTVY
TADFSQNSAAGTADIIRRINSGLAANPNVCYILQGYSQGAAATVVALQQLGTSGPAFNAVKGVF
LIGNPDHKSGLTCNVDSNGGTTTRNVNGLSVAYQGSVPSGWVSKTLDVCAYGDGVCDTAHG
FGINAQHLSYPSDQGVQTMGYKFAVNKLGGSA
SEQ ID NO: 33: CLE1J
APTPESAEAHELEARATSSACPQYVLINTRGTGEPQGQSAGFRTMNSQITAALSGGTIYNTVY
TADFSQNSAAGTADIIRRINSGLAANPNVCYILQGYSQGAAATVVALQQLGTSGAAFNAVKGVF
LIGNPDHKSGLTCNVDSNGGITTRNVNGLSVAYQGSVPSGWVSKTLDVCAYGDGVCDTAHGF
GINAQHLSYPSDQGVQTMGYKFAVNKLGGSA
SEQ ID NO: 34: CLE1K
APTPESAEAHELEARATSSACPQYVLINTRGTGEPQGQSAGFRTMNSQITAALSGGTIYNTVY
TADFSQNSAAGTADIIRRINSGLAANPNVCYILQGYSQGAAATVVALQQLGTSGAAFNAVKGVF
LIGNPDHKSGLTCNVDSNGGTTTRNVNGLSVAYQGSVPSGWVSKTLDVCAYGDGVCDTAHG
FGINAQHLSYPSDEGVQTMGYKFAVNKLGGSA
SEQ ID NO: 35: CLE1L
APTPESAEAHELEARATSSACPQYVLINTRGTGEPQGQSVGFRTMNSQITAALSGGTIYNTVY
TADFSQNSAAGTADIIRRINSGLAANPNVCYILQGYSQGAAATVVALQRLGTSGAAFNAVKGVF
LIGNPDHKSGLTCNVDSNGGTTTRNVNGLSVAYQGSVPSGWVSKTLDVCAYGDGVCDTAHG
FGVNAQHLSYPSDQGVQTMGYKFAVNKLGGSA

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. An enzyme comprising an amino acid sequence having 90% or greater identity to the amino acid sequence set forth in SEQ ID NO:4, wherein the enzyme comprises amino acid substitutions at positions Q1, G3, T29, and E47, according to the numbering of SEQ ID NO:4.

2. The enzyme of claim 1, wherein the amino acid at position Q1 is arginine.

3. The enzyme of claim 1, wherein the amino acid substitution at position Q1 is Q1A, Q1C, Q1D, Q1E, Q1F, Q1G, Q1H, Q1K, Q1L, Q1M, Q1N, Q1P, Q1S, Q1T, Q1V, or Q1Y.

4. The enzyme of any one of claims 1 to 3, wherein the amino acid at position G3 is serine.

5. The enzyme of any one of claims 1 to 3, wherein the amino acid substitution at position G3 is G3A, G3C, G3D, G3E, G3F, G3H, G31, G3L, G3N, G3Q, G3R, G3T, G3V, or G3Y.

6. The enzyme of any one of claims 1 to 5, wherein the amino acid at position T29 is lysine.

7. The enzyme of any one of claims 1 to 6, wherein the amino acid at position E47 is glutamine.

8. The enzyme of any one of claims 1 to 6, wherein the amino acid substitution at position E47 is E47A, E47C, E47F, E47G, E47H, E471, E47L, E47M, E47N, E47R, E47S, E47T, E47V, or E47Y.

9. The enzyme of claim 1, wherein the enzyme comprises amino acid substitutions Q1R, G3S, T29K, and E470.

10. The enzyme of any one of claims 1 to 9, wherein the enzyme comprises an amino acid substitution at A4, S11, N15, T37, T164, or T166, or any combination of two or more thereof, wherein numbering is as in SEQ ID NO:4.

11. A nucleic acid encoding the enzyme of any one of claims 1 to 10.

12. A cell comprising the nucleic acid of claim 11.

13. The cell of claim 12, wherein the nucleic acid is present in an expression vector.

14. A method of producing the enzyme of any one of claims 1 to 10, comprising culturing the cell of claim 13 under conditions suitable for expression of the enzyme, wherein the enzyme is produced.

15. A method of degrading plastic, the method comprising contacting the plastic with the enzyme of any one of claims 1 to 10.

16. The method of claim 15, wherein the plastic is a polyester.

17. The method of claim 16, wherein the polyester is a polyethylene terephthalate (PET).

18. The method of any one of claims 14 to 17, wherein the plastic is present in a fiber, fabric, or textile.

19. An enzyme comprising an amino acid sequence having 90% or greater identity to the amino acid sequence set forth in SEQ ID NO:3, wherein the enzyme comprises amino acid substitutions at positions A40, Q112 and 1191, according to the numbering of SEQ ID NO:3.

20. The enzyme of claim 19, wherein the amino acid at position A40 is valine.

21. The enzyme of claim 19 or claim 20, wherein the amino acid at position Q112 is arginine.

22. The enzyme of any one of claims 19 to 21, wherein the amino acid at position 1191 is valine.

23. The enzyme of claim 19, wherein the enzyme comprises amino acid substitutions A40V, Q112R, and 1191V.

24. The enzyme of any one of claims 19 to 23, wherein the enzyme further comprises an amino acid substitution at positions Y94, Q111, A118, T148, or Q202, or any combination of two or more thereof, wherein numbering is as in SEQ ID NO:3.

25. The enzyme of claim 24, wherein the enzyme comprises the amino acid substitution Y94F, Q111K, Al18P, T1481, or Q202E, or any combination of two or more thereof.

26. A nucleic acid encoding the enzyme of any one of claims 19 to 25.

27. A cell comprising the nucleic acid of claim 26.

28. The cell of claim 27, wherein the nucleic acid is present in an expression vector.

29. A method of producing the enzyme of any one of claims 19 to 25, comprising culturing the cell of claim 27 under conditions suitable for expression of the enzyme, wherein the enzyme is produced.

30. A method of degrading plastic, the method comprising contacting the plastic with the enzyme of any one of claims 19 to 25.

31. The method of claim 30, wherein the plastic is a polyester.

32. The method of claim 31, wherein the polyester is a polylactic acid.

33. The method of any one of claims 30 to 32, wherein the plastic is present in a fiber, fabric, or textile.


SEQUENCE LISTING (35 sequences)

SEQ ID NO: 1 - DNA, 660 bp, Cryptococcus sp.
  source 1..660: mol_type = unassigned DNA
gcgccgactcctgaaagcgccgaggctcatgagctggaagctagggccacttcatccgcttgtccacaatacgttttgataaacaccaggggcacaggagaaccacaggggcaatcagccggcttcaggaccatgaatagtcagattaccgcagcattgtcagggggcaccatatataatacagtttataccgcggatttctcacagaacagtgccgcggggacagcagacataattcgtaggatcaatagcggtttagcagcgaatcccaatgtctgttatattctacaggggtactcacagggagcggccgcgaccgtcgttgcgttacaacaactaggcacctccggggctgcattcaacgccgttaaaggagtcttccttatcgggaacccggaccacaaatctgggcttacgtgcaatgtcgatagcaatggtgggaccaccacccgtaatgtcaatggcttgtcagtcgcttaccaaggctcagtgccttcaggttgggtctcaaagactttggacgtatgtgcctatggggatggcgtgtgcgatactgcacatgggtttggtatcaatgcccaacatttatcttatccttcagaccagggagtccagacaatgggctacaaatttgcggtcaacaaattgggcggtagtgct

SEQ ID NO: 2 - DNA, 582 bp, Humicola insolens
  source 1..582: mol_type = unassigned DNA
caactaggtgctatcgaaaatgggttagagagcggctccgcaaatgcttgtcccgatgctatattgatctttgcaaggggaacaacggaacccggtaacatgggaattttagttggccctgcacttgcgaatgggttagaatcacacattcgtaatatttggatacaaggcgtagggggaccctacgatgctgctctagccacaaacttcctgcctcgtggcaccagtcaagccaacattgacgaaggaaagagattgtttgcgcttgctaaccagaaatgtcccaatacccccgttgttgcaggtggttattcacagggcgcagcacttattgctgcggcggtctccgagttatctggggcagtgaaggagcaagttaaaggcgtgactttgtttggatatacccagaatttacaaaatatgggcggcatcccaaattaccctcgtgaacgtacaaaggtcttctgcaacgccggcgatgccgtgtgcactggcacgctgataattacccctgcacacctatcatacacaatagaggctagaggcgaggcggcacgtttcttgagggacagaatacgtgca

SEQ ID NO: 3 - protein, 220 aa, Cryptococcus sp.
APTPESAEAHELEARATSSACPQYVLINTRGTGEPQGQSAGFRTMNSQITAALSGGTIYNTVYTADFSQNSAAGTADIIRRINSGLAANPNVCYILQGYSQGAAATVVALQQLGTSGAAFNAVKGVFLIGNPDHKSGLTCNVDSNGGTTTRNVNGLSVAYQGSVPSGWVSKTLDVCAYGDGVCDTAHGFGINAQHLSYPSDQGVQTMGYKFAVNKLGGSA

SEQ ID NO: 4 - protein, 194 aa, Humicola insolens
QLGAIENGLESGSANACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCTGTLIITPAHLSYTIEARGEAARFLRDRIRA

SEQ ID NO: 5 - protein, 239 aa, Saitozyma flava
MLFSAIALAALSAASMSCAAPTPESAAGHELEARATSSACPQYVLINTRGTGEPQGQSAGFRTMNSQITAALSGGTIYNTVYTADFSQNSAAGTADIIRRINSGLASNPNVCYILQGYSQGAAATVVALQQLGTSGAAFNAVKGVFLIGNPDHKAGLTCNVDSNGGTTTRNVNGLSVAYQGSIPSGWVSKTLDVCAYGDGVCDTTHGYGINAPHLSYPNDQGVQSMGYKFAVNKLGGSA

SEQ ID NO: 6 - protein, 194 aa, synthetic construct
ILGAIENGLESGSANACPDAILIFARGSTEPGNMGILVGPALANGLRSHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCTGTLIITPAHLSYTIEARGEAARFLRDRIRA

SEQ ID NO: 7 - protein, 194 aa, synthetic construct
ILSAIENGLESGSANACPDAILIFARGSTEPGNMGIIVGPALANGLRSHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCTGTLIITPAHLSYTIEARGEAARFLRDRIRA

SEQ ID NO: 8 - protein, 194 aa, synthetic construct
RLTAIENGLESGSANACPDAILIFARGSTEPGNMGILVGPALANGLRSHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCTGTLIITPAHLSYTIEARGEAARFLRDRIRA

SEQ ID NO: 9 - protein, 194 aa, synthetic construct
RLSAIENGLESGSANACPDAILIFARGSKEPGNMGITVGPALANGLQSHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCTGTLIITPAHLSYTIEARGEAARFLRDRIRA

SEQ ID NO: 10 - protein, 194 aa, synthetic construct
QLGAIENGLENGSADACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCSGTLIITPAHLSYTIEARGEAARFLRDRIRA

SEQ ID NO: 11 - protein, 194 aa, synthetic construct
QLGVIENGLESGSADACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCSGTLIITPAHLSYTIEARGEAARFLRDRIRA

SEQ ID NO: 12 - protein, 194 aa, synthetic construct
QLGVIENGLENGSANACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCSGTLIITPAHLSYTIEARGEAARFLRDRIRA

SEQ ID NO: 13 - protein, 194 aa, synthetic construct
QLGVIENGLENGSADACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCTGTLIITPAHLSYTIEARGEAARFLRDRIRA

SEQ ID NO: 14 - protein, 194 aa, synthetic construct
QLGVIENGLENGSANACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCTGTLIITPAHLSYTIEARGEAARFLRDRIRA

SEQ ID NO: 15 - protein, 194 aa, synthetic construct
QLGVIENGLESGSADACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCTGTLIITPAHLSYTIEARGEAARFLRDRIRA

SEQ ID NO: 16 - protein, 194 aa, synthetic construct
QLGVIENGLESGSANACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCSGTLIITPAHLSYTIEARGEAARFLRDRIRA

SEQ ID NO: 17 - protein, 194 aa, synthetic construct
QLGAIENGLENGSADACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCTGTLIITPAHLSYTIEARGEAARFLRDRIRA

SEQ ID NO: 18 - protein, 194 aa, synthetic construct
QLGAIENGLENGSANACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCSGTLIITPAHLSYTIEARGEAARFLRDRIRA

SEQ ID NO: 19 - protein, 194 aa, synthetic construct
QLGAIENGLESGSADACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCSGTLIITPAHLSYTIEARGEAARFLRDRIRA

SEQ ID NO: 20 - protein, 194 aa, synthetic construct
QLGVIENGLESGSANACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCTGTLIITPAHLSYTIEARGEAARFLRDRIRA

SEQ ID NO: 21 - protein, 194 aa, synthetic construct
QLGAIENGLENGSANACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCTGTLIITPAHLSYTIEARGEAARFLRDRIRA

SEQ ID NO: 22 - protein, 194 aa, synthetic construct
QLGAIENGLESGSADACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCTGTLIITPAHLSYTIEARGEAARFLRDRIRA

SEQ ID NO: 23 - protein, 197 aa, synthetic construct
QLGAIENGLESGSANACPDAILIFARGSTEPGNMGITVGPALANGLESHIRNIWIQGVGGPYDAALATNFLPRGTSQANIDEGKRLFALANQKCPNTPVVAGGYSQGAALIAAAVSELSGAVKEQVKGVALFGYTQNLQNRGGIPNYPRERTKVFCNVGDAVCSGTLIITPAHLSYTIEARGEAARFLRDRIRASEQ

SEQ ID NO: 24 - protein, 220 aa, synthetic construct
APTPESAEAHELEARATSSACPQYVLINTRGTGEPQGQSAGFRTMNSQITAALSGGTIYNTVYTADFSQNSAAGTADIIRRINSGLAANPNVCFILQGYSQGAAATVVALKQLGTSGPAFNAVKGVFLIGNPDHKSGLTCNVDSNGGITTRNVNGLSVAYQGSVPSGWVSKTLDVCAYGDGVCDTAHGFGINAQHLSYPSDEGVQTMGYKFAVNKLGGSA

SEQ ID NO: 25 - protein, 220 aa, synthetic construct
APTPESAEAHELEARATSSACPQYVLINTRGTGEPQGQSAGFRTMNSQITAALSGGTIYNTVYTADFSQNSAAGTADIIRRINSGLAANPNVCFILQGYSQGAAATVVALKQLGTSGPAFNAVKGVFLIGNPDHKSGLTCNVDSNGGITTRNVNGLSVAYQGSVPSGWVSKTLDVCAYGDGVCDTAHGFGINAQHLSYPSDQGVQTMGYKFAVNKLGGSA

SEQ ID NO: 26 - protein, 220 aa, synthetic construct
APTPESAEAHELEARATSSACPQYVLINTRGTGEPQGQSAGFRTMNSQITAALSGGTIYNTVYTADFSQNSAAGTADIIRRINSGLAANPNVCYILQGYSQGAAATVVALKQLGTSGPAFNAVKGVFLIGNPDHKSGLTCNVDSNGGITTRNVNGLSVAYQGSVPSGWVSKTLDVCAYGDGVCDTAHGFGINAQHLSYPSDEGVQTMGYKFAVNKLGGSA

SEQ ID NO: 27 - protein, 220 aa, synthetic construct
APTPESAEAHELEARATSSACPQYVLINTRGTGEPQGQSAGFRTMNSQITAALSGGTIYNTVYTADFSQNSAAGTADIIRRINSGLAANPNVCFILQGYSQGAAATVVALQQLGTSGPAFNAVKGVFLIGNPDHKSGLTCNVDSNGGITTRNVNGLSVAYQGSVPSGWVSKTLDVCAYGDGVCDTAHGFGINAQHLSYPSDEGVQTMGYKFAVNKLGGSA

SEQ ID NO: 28 - protein, 220 aa, synthetic construct
APTPESAEAHELEARATSSACPQYVLINTRGTGEPQGQSAGFRTMNSQITAALSGGTIYNTVYTADFSQNSAAGTADIIRRINSGLAANPNVCFILQGYSQGAAATVVALKQLGTSGAAFNAVKGVFLIGNPDHKSGLTCNVDSNGGITTRNVNGLSVAYQGSVPSGWVSKTLDVCAYGDGVCDTAHGFGINAQHLSYPSDEGVQTMGYKFAVNKLGGSA

SEQ ID NO: 29 - protein, 220 aa, synthetic construct
APTPESAEAHELEARATSSACPQYVLINTRGTGEPQGQSAGFRTMNSQITAALSGGTIYNTVYTADFSQNSAAGTADIIRRINSGLAANPNVCFILQGYSQGAAATVVALKQLGTSGPAFNAVKGVFLIGNPDHKSGLTCNVDSNGGTTTRNVNGLSVAYQGSVPSGWVSKTLDVCAYGDGVCDTAHGFGINAQHLSYPSDEGVQTMGYKFAVNKLGGSA

SEQ ID NO: 30 - protein, 220 aa, synthetic construct
APTPESAEAHELEARATSSACPQYVLINTRGTGEPQGQSAGFRTMNSQITAALSGGTIYNTVYTADFSQNSAAGTADIIRRINSGLAANPNVCFILQGYSQGAAATVVALQQLGTSGAAFNAVKGVFLIGNPDHKSGLTCNVDSNGGTTTRNVNGLSVAYQGSVPSGWVSKTLDVCAYGDGVCDTAHGFGINAQHLSYPSDQGVQTMGYKFAVNKLGGSA

SEQ ID NO: 31 - protein, 220 aa, synthetic construct
APTPESAEAHELEARATSSACPQYVLINTRGTGEPQGQSAGFRTMNSQITAALSGGTIYNTVYTADFSQNSAAGTADIIRRINSGLAANPNVCYILQGYSQGAAATVVALKQLGTSGAAFNAVKGVFLIGNPDHKSGLTCNVDSNGGTTTRNVNGLSVAYQGSVPSGWVSKTLDVCAYGDGVCDTAHGFGINAQHLSYPSDQGVQTMGYKFAVNKLGGSA

SEQ ID NO: 32 - protein, 220 aa, synthetic construct
APTPESAEAHELEARATSSACPQYVLINTRGTGEPQGQSAGFRTMNSQITAALSGGTIYNTVYTADFSQNSAAGTADIIRRINSGLAANPNVCYILQGYSQGAAATVVALQQLGTSGPAFNAVKGVFLIGNPDHKSGLTCNVDSNGGTTTRNVNGLSVAYQGSVPSGWVSKTLDVCAYGDGVCDTAHGFGINAQHLSYPSDQGVQTMGYKFAVNKLGGSA

SEQ ID NO: 33 - protein, 220 aa, synthetic construct
APTPESAEAHELEARATSSACPQYVLINTRGTGEPQGQSAGFRTMNSQITAALSGGTIYNTVYTADFSQNSAAGTADIIRRINSGLAANPNVCYILQGYSQGAAATVVALQQLGTSGAAFNAVKGVFLIGNPDHKSGLTCNVDSNGGITTRNVNGLSVAYQGSVPSGWVSKTLDVCAYGDGVCDTAHGFGINAQHLSYPSDQGVQTMGYKFAVNKLGGSA

SEQ ID NO: 34 - protein, 220 aa, synthetic construct
APTPESAEAHELEARATSSACPQYVLINTRGTGEPQGQSAGFRTMNSQITAALSGGTIYNTVYTADFSQNSAAGTADIIRRINSGLAANPNVCYILQGYSQGAAATVVALQQLGTSGAAFNAVKGVFLIGNPDHKSGLTCNVDSNGGTTTRNVNGLSVAYQGSVPSGWVSKTLDVCAYGDGVCDTAHGFGINAQHLSYPSDEGVQTMGYKFAVNKLGGSA

SEQ ID NO: 35 - protein, 220 aa, synthetic construct
APTPESAEAHELEARATSSACPQYVLINTRGTGEPQGQSVGFRTMNSQITAALSGGTIYNTVYTADFSQNSAAGTADIIRRINSGLAANPNVCYILQGYSQGAAATVVALQRLGTSGAAFNAVKGVFLIGNPDHKSGLTCNVDSNGGTTTRNVNGLSVAYQGSVPSGWVSKTLDVCAYGDGVCDTAHGFGVNAQHLSYPSDQGVQTMGYKFAVNKLGGSA